(19) United States
(12) Patent Application Publication
(10) Pub. No.: US 2014/0194512 A1
(43) Pub. Date: Jul. 10, 2014

(54) COMPOSITIONS COMPRISING DOCOSAPENTAENOIC ACID AND METHODS OF USE

(71) Applicant: Matinas BioPharma, Inc., Bedminster, NJ (US)

(72) Inventors: Abdel Aziz FAWZY, Boynton Beach, FL (US); George BOBOTAS, Tarpon Springs, FL (US); Ihor TERLECKYJ, Berwyn, PA (US); Roelof RONGEN, Califon, NJ (US)

(73) Assignee: Matinas BioPharma, Inc., Bedminster, NJ (US)

(21) Appl. No.: 14/207,099

(22) Filed: Mar. 12, 2014

Related U.S. Application Data
(63) Continuation-in-part of application No. 14/109,338, filed on Dec. 17, 2013.
Application No. 14/109,338 is a continuation-in-part of application No. PCT/US13/46176, filed on Jun. 17, 2013.
(60) Provisional application No. 61/780,948, filed on Mar. 13, 2013.
(60) Provisional application No. 61/734,331, filed on Dec. 6, 2012.
(60) Provisional application No. 61/660,757, filed on Jun. 17, 2012.

(51) Int. Cl. A61K 31/202

(52) U.S. Cl. CPC A61K 31/202
USPC 514/560

(57) ABSTRACT

Orally administrable composition comprising fatty acids comprising docosapentaenoic acid (DPA) and docosahexaenoic acid (DHA). The compositions can be used for the treatment or prophylaxis of a variety of conditions, including liver-related conditions.

### RELATED APPLICATIONS

This application is a continuation-in-part (CIP) application of U.S. patent application Ser. No. 14/109,338, filed on Dec. 17, 2013, which is a continuation-in-part (CIP) application of PCT International Application No. PCT/US13/46176, filed on Jun. 17, 2013, which claims the benefit of U.S. Provisional Patent Application No. 61/660,757, filed Jun. 17, 2012, U.S. Provisional Patent Application No. 61/734,331, filed Dec. 6, 2012, and U.S. Provisional Patent Application No. 61/780,948, filed Mar. 13, 2013, the contents of which are incorporated herein by reference.

### FIELD OF INVENTION

The present invention relates to omega-3 fatty acid compositions, and methods of treating, preventing, reducing the occurrence of, and improving symptoms associated with liver-related conditions.

### BACKGROUND OF THE INVENTION

Marine oils, also commonly referred to as fish oils, are a good source of the two main omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides (TG), increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

The table directly below lists the most common omega-3 fatty acids, including their 3-letter abbreviation code. In this application, the use of any of the 3-letter abbreviations shall refer to the omega-3 fatty acid, unless otherwise indicated (e.g. DPA or DPA 22:5 (n-3) or DPA 22:5-n3 or DPA 22:5n3 or DPA-n3, which all refer to the omega-3 isomer of docosapentaenoic acid).

| Common Name for Omega-3 | Codified | |
|---|---|---|
| Fatty Acid (+abbreviation) | Lipid Name | Chemical Name |
| Hexadecatrienoic acid (HTA) | 16:3 (n-3) | all-cis-7,10,13-hexadecatrienoic acid |
| α-Linolenic acid (ALA) | 18:3 (n-3) | all-cis-9,12,15-octadecatrienoic acid |
| Stearidonic acid (SDA) | 18:4 (n-3) | all-cis-6,9,12,15-octadecatetraenoic acid |
| Eicosatrienoic acid (ETE) | 20:3 (n-3) | all-cis-11,14,17-eicosatrienoic acid |
| Eicosatetraenoic acid (ETA) | 20:4 (n-3) | all-cis-8,11,14,17-eicosatetraenoic acid |
| Eicosapentaenoic acid (EPA) | 20:5 (n-3) | all-cis-5,8,11,14,17-eicosapentaenoic acid |
| Heneicosapentaenoic acid (HPA) | 21:5 (n-3) | all-cis-6,9,12,15,18-heneicosapentaenoic acid |
| Docosapentaenoic acid (DPA) or | 22:5 (n-3) | all-cis-7,10,13,16,19-docosapentaenoic acid |
| Clupanodonic acid | | |
| Docosahexaenoic acid (DHA) | 22:6 (n-3) | all-cis-4,7,10,13,16,19-docosahexaenoic acid |
| Tetracosapentaenoic acid (TPA) | 24:5 (n-3) | all-cis-9,12,15,18,21-tetracosapentaenoic acid |
| Tetracosahexaenoic acid (THA) or | 24:6 (n-3) | all-cis-6,9,12,15,18,21-tetracosahexaenoic acid |
| Nisinic acid | | |

One form of omega-3 fatty acids is a concentrate of omega-3, long chain, polyunsaturated fatty acids from fish oil containing DHA ethyl esters, EPA ethyl esters as well as ethyl esters of other omega-3 fatty acids (described in USP35 for LOVAZA®) and is sold under the trademarks OMACOR® and LOVAZA®. Such a form of omega-3 fatty acid comprises at least 90% omega-3 fatty acids of which at least 80% EPA+DHA (in a ratio of 1.2:1) and is described, for example, in U.S. Pat. Nos. 5,502,077, 5,656,667 and 5,698,594. LOVAZA® (omega-3-acid ethyl esters) is indicated for the treatment of patients with hypertriglyceridemia with TG levels of 500 mg/dL or higher.

Another form of omega-3 fatty acid concentrate is sold under the trademark EPADEL® for the treatment of dyslipidemia. This product is described as 98% EPA ethyl ester in Lancet (Vol. 369; Mar. 31, 2007; 1090-1098) reporting on a large outcome study with EPADEL®. EPADEL® is known to contain less than 1% of any fatty acid other than EPA.

Similar to EPADEL®, another form of omega-3 fatty acid concentrate also consists almost entirely of EPA ethyl ester and is known under its developmental stage name AMR101 or its trade name VASCEPA®. This product is described in US patent application 2010/0278879 as comprising at least 95% EPA (typically referred to as 97% or at least 96% in company releases and references) and less than 1% of any other fatty acid. AMR101 was previously under development for the treatment of Huntingdon's Disease but failed in phase III clinical development. Subsequently, AMR101 was entered in a development program for hypertriglyceridemia and mixed dyslipidemia.

Yet another concentrate of omega-3, long chain, polyunsaturated fatty acids from fish oil containing approximately 75% DHA and EPA as free fatty acids is known under its developmental stage name EPANOVA™. This product is described as comprising approximately 55% EPA and 20% DHA. EPANOVA™ was previously under development for the treatment of Crohn's Disease but failed in phase III clinical development. Subsequently, EPANOVA™ was entered in a development program for hypertriglyceridemia and mixed dyslipidemia.

Generally, the bioavailability and therapeutic effect of omega-3 fatty acid compositions is dose dependent, i.e. the higher the dose, the greater the therapeutic affect and bioavailability. However, the effect of each specific omega-3 fatty acid composition may be different, and therefore the level of therapeutic effect of one composition at a given dose cannot necessarily be inferred from the level of therapeutic effects of other omega-3 fatty acid compositions at the same or similar dose.

Omega-3 fatty acids are known to be “essential fatty acids”. There are two series of essential fatty acids (EFAs) in humans. They are termed “essential” because they cannot be synthesized de novo in mammals. These fatty acids can be interconverted within a series, but the omega-6 (n-6) series cannot be converted to the omega-3 series nor can the omega-3 (n-3) series be converted to the omega-6 series in humans. The main EFAs in the diet are linoleic acid of the omega-6 series and alpha-linolenic acid of the omega-3 series. However, to fulfill most of their biological effects these “parent” EFAs must be metabolised to the other longer chain fatty acids. Each fatty acid probably has a specific role in the body. The scientific literature suggests that particularly important in the n-6 series are dihomogammalinolenic acid (DGLA, 20:3-n6) and arachidonic acid (ARA, 20:4-n6), while particularly important in the n-3 series are eicosapentaenoic acid (EPA, 20:5-n3) and docosahexaenoic acid (DHA, 22:6-n3).

U.S. Pat. No. 6,479,544 describes an invention in which it is found that ARA is highly desirable rather than undesirable and it may be helpful to administer ARA in association with EPA. This invention provides pharmaceutical formulations containing eicosapentaenoic acid or any appropriate derivative (hereinafter collectively referred to as EPA) and arachidonic acid (ARA), as set out in the granted claims for this patent. ARA may be replaced by one or more of its precursors, DGLA or GLA. In this reference, the ratio of EPA to ARA is preferably between 1:1 and 20:1.

Patent application PCT/GB 2004/000242 describes the treatment or prevention of psoriasis with a formulation comprising more than 95% EPA and less than 2% DHA. In another embodiment of this invention the EPA is replaced with DPA.

Patent application PCT/NL 2006/050291 (WO/2007/058538, GB 0301701.9) describes combinations of idigestible oligosaccharides and long chain polyunsaturated fatty acids such as ARA, EPA, DA, and combinations thereof to improve intestinal barrier integrity, improving barrier function, stimulating gut maturation and/or reducing intestinal barrier permeability.

Lindeborg et al. (Prostag Leukotr Ess, 2013, 88:313-319) discloses a study evaluating postprandial metabolism of docosapentaenoic acid (DPA) and eicosapentaenoic acid (EPA) in humans.

Holub et al. (Lipids. 2011, 46:399-407) discloses a study assessing the effect of oral supplementation with docosapentaenoic acid (DPA) on levels of serum and tissue lipid classes and their fatty acid compositions in rat liver, heart, and kidney.

Non-alcoholic fatty liver disease (NAFLD) is thought to be the most common chronic liver disease. The prevalence of NAFLD, on a global level, ranges from roughly 15% to 30%, and is associated with multiple variables. In the United States, estimates of NAFLD prevalence are in the range of 24%. In Hispanics, this rate is higher than both European-origin and Afro-American origin genotypes. Of all the liver transplants in the United States, it has been estimated that close to 10% are a result of non-alcoholic steatohepatitis (NASH), which is the most extreme form of NAFLD. In addition, of all the cardiovascular events resulting in death, NAFLD co-morbidity is thought to occur in 24% of all cases. Other co-morbidity factors associated with NAFLD and NASH include obesity, metabolic syndrome and type 2 diabetes. As the incidence rate for all these disease states is currently rising, it is the opinion of those skilled in the art that the incidence and prevalence of NAFLD, NASH, and its consequences will be increasing in the future (AASLD/IASL NAFLD Symposium, 2013, Washington. D.C.).

There is a high unmet need in the area of liver conditions, especially fatty liver and more specifically NAFLD and its more severe form NASH. NAFLD may be considered as a precursor to NASH, in the spectrum of disease progression. NAFLD is characterized by high abnormal levels of lipids, primarily in the form of triglycerides, cholesterol and fatty acids, that are elevated and/or stored in the liver (simple steatosis). With prolonged time, the pathophysiology of disease progression can result in the process of fibrosis and necroinflammation, giving rise to the state of NASH. As this particular process continues, the level of ensuing fibrosis increases, as does an inflammatory response, that decreases normal liver functioning which can ultimately result in cirrhosis, liver failure and/or death. There is also a high risk of co-morbidity for liver cancer associated with NAFLD, and especially NASH.

Diagnosis of early NAFLD relies primarily on recognition of elevated liver enzymes, such as ALT, AST and perhaps GGT. In those skilled in the art, elevations of these enzymes may precipitate the need for further tests in the form of imaging, including ultrasound and MRI, or related tests such as but not limited to FIBROTEST®. For the diagnosis of NASH, the current gold standard is histopathological evaluation of the degree of fibrosis determined by examining liver samples obtained thru punch biopsy. Other non-invasive methods for determining fibrosis include determination of liver stiffness by specific ultrasound techniques, and are being evaluated as surrogates and/or replacement for performing liver biopsies.

For NAFLD, the main objective is to decrease lipid levels in the liver. The first course of therapy is life style modification, mainly by dietary means and a weight loss program. In the instance of NASH, the main objective is to curtail further development of fibrosis. There are currently limited choices of therapeutic agents for the treatment of NASH. In adults, the primary agents that could be used include vitamin E and pentoxyfiline. However, there are limitations to the use of these agents. In the instance of vitamin E, long term use is associated with an increased risk for cardiovascular events, including myocardial infarct and cardiac death.

For instances of liver failure, the only viable alternative is liver transplantation, which is costly, subject to rejection, and predisposes the patient to increased risk of opportunistic microbial infections due to the use of immunosuppressive agents. In addition, the availability and suitability of donor livers is highly limited, which would decrease chance of survival for persons in the state of liver failure. Therefore, there is a need in the art for an effective therapy for liver-related conditions.

HMG-CoA reductase is a rate controlling enzyme of the mevalonate pathway. It is a transmembrane protein which catalyzes a key step in the mevalonate pathway, which is involved in the synthesis of steroid, isoprenoids, and other lipids. In humans, HMG-CoA reductase is the rate limiting step in cholesterol synthesis and is a major drug target for cholesterol reducing drugs like “statins,” or HMG-CoA reductase inhibitors.

Proprotein convertase subtilisin/kexin type 9, also known as PCSK9, is an enzyme that in humans is encoded by the PCSK9 gene. PCSK9 is thought to act in cholesterol homeostasis, and drugs that block PCSK9 are thought to be effective in lowering cholesterol, including low-density lipoprotein cholesterol (LDL-D).

All references cited herein are incorporated by reference in their entirety.

### SUMMARY OF THE INVENTION

The present invention provides omega-3 fatty acid compositions and methods of administering these compositions.

The present invention provides a pharmaceutical composition comprising docosapentaenoic acid (DPA) and docosahexaenoic acid (DHA) in an amount of at least 60% of total amount of the fatty acids present in the composition, wherein the ratio of DPA to DHA (DPA:DHA) is between about 10:1 to about 1:10.

The present invention provides methods comprising administering the compositions. The present invention provides a method of treating, preventing, reducing the occurrence of, and improving symptoms associated with liver-related conditions. Examples of liver-related conditions include, but are not limited to fatty liver, non-alcoholic steatohepatitis (NASH), non-alcoholic fatty liver disease (NAFLD), alcoholic steatohepatitis (ASH), hepatitis including but not limited to hepatitis C, HIV (human immunodeficiency virus) infection and any liver syndrome arising from the treatment of HIV, drug-induced fatty liver or sequalae (including but not limited to NAFLD, NASH), liver failure, liver transplantation, transplanted liver failure, liver damage associated with other organ system afflictions including but not limited to renal failure or disease, abnormally elevated liver enzymes (such as ALT, AST and/or GGT) associated with disease (such as diabetes type 2), or drug-induced afflictions. The present invention further provides a method of reducing expression of HMG-CoA reductase and/or reducing levels of HMG-CoA reductase in a subject. The present invention further provides a method of reducing the expression of proprotein convertase subtilisin/kexin type 9 (PCSK9) and/or reducing levels of PCSK9 in a subject.

### DETAILED DESCRIPTION OF THE INVENTION

The present invention provides an orally administrable pharmaceutical composition comprising fatty acids. In some embodiments of the present invention, the pharmaceutical composition comprises docosapentaenoic acid (DPA) and docosahexaenoic acid (DHA) in an amount of at least 60% of total amount of the fatty acids present in the composition. In some embodiments, the pharmaceutical composition comprises DPA and DHA in an amount of about 65% or greater, alternatively about 70% or greater, alternatively about 75% or greater, alternatively about 80% or greater, alternatively about 80% or greater, alternatively about 81% or greater, alternatively about 82% or greater, alternatively about 83% or greater, alternatively about 84% or greater, alternatively about 85% or greater, alternatively about 86% or greater, alternatively about 87% or greater, alternatively about 88% or greater, alternatively about 89% or greater, alternatively about 90% or greater, alternatively about 91% or greater, alternatively about 92% or greater, alternatively about 93% or greater, alternatively about 94% or greater, alternatively about 95% or greater, alternatively about 96% or greater, alternatively about 97% or greater, alternatively about 98% or greater, or alternatively about 99% or greater of the total amount of fatty acids present in the composition. In some embodiments, the pharmaceutical composition comprises DPA and DHA in an amount of between about 70% to about 99%, alternatively about 75% to about 99%, alternatively about 80% to about 95%, of the total amount of the fatty acids present in the composition.

In some embodiments, the compositions comprise docosapentaenoic acid (DPA) and docosahexaenoic acid (DHA) such that the DPA:DHA ratio in the composition is about 10:1 to 1:10, alternatively about 9:1 to about 1:9, alternatively about 8:1 to 1:8, alternatively about 7:1 to about 1:7, alternatively about 6:1 to about 1:6, alternatively about 5:1 to about 1:5, or alternatively about 4:1 to about 1:4.

In some embodiments, the composition comprises DPA and DHA in a ratio of DPA:DHA of at least 2:1. In some embodiments, the composition comprises DPA:DHA in a ratio of about 2:1 to about 10:1, alternatively about 2:1 to about 9:1, alternatively about 2:1 to about 8:1, alternatively about 2:1 to about 7:1, alternatively about 2:1 to about 6:1, alternatively about 2:1 to about 5:1, alternatively about 2:1 to about 4:1, alternatively about 3:1 to about 5:1, or alternatively about 4:1.

In some embodiments, the composition comprises DPA and DHA in a ratio of DPA:DHA of about 5:1 to about 1:5, alternatively about 4:1 to about 1:4, alternatively about 3:1 to about 1:3, alternatively about 2:1 to about 1:2, or alternatively about 1:1.

In some embodiments, the composition comprises DHA (docosahexaenoic acid) and DPA (docosahexaenoic acid) in a ratio of DHA:DPA of about least 2:1. In some embodiments, the composition comprises DHA:DPA in a ratio of about 2:1 to about 10:1, alternatively about 2:1 to about 9:1, alternatively about 2:1 to about 8:1, alternatively about 2:1 to about 7:1, alternatively about 2:1 to about 6:1, alternatively about 2:1 to about 5:1, alternatively about 2:1 to about 4:1, alternatively about 3:1 to about 5:1, or alternatively about 4:1.

In other embodiments, a relatively small amount of DHA as compared to DPA is present. In these embodiments, the compositions of the present invention comprise no more than 15:1 of DHA:DPA, alternatively no more than 12:1 of DHA:DPA, alternatively no more than 10:1 of DHA:DPA, alternatively no more than 8:1 of DHA:DPA, alternatively no more than 5:1 of DHA:DPA, alternatively no more than 3:1 of DHA:DPA, alternatively no more than 2:1 of DHA:DPA, alternatively no more than 1:1 of DHA:DPA, alternatively no more than 1:2 of DHA:DPA, alternatively no more than 1:3 of DHA:DPA, alternatively no more than 1:4 of DHA:DPA, alternatively no more than 1:5 of DHA:DPA, alternatively no more than 1:6 of DHA:DPA, alternatively no more than 1:7 of DHA:DPA, alternatively no more than 1:8 of DHA:DPA, alternatively no more than 1:10 of DHA:DPA, alternatively no more than 1:12 of DHA:DPA, alternatively no more than 1:15 of DHA:DPA, alternatively no more than 1:20 of DHA:DPA, alternatively no more than 1:25 of DHA:DPA, alternatively no more than 1:50 of DHA:DPA, alternatively no more than 1:75 of DHA:DPA, alternatively no more than 1:90 of DHA:DPA, alternatively no more than 1:95 of DHA:DPA, alternatively no more than 1:100 of DHA:DPA. In some embodiments, the ratio of DHA:DPA is less than 2:1.

The docosapentaenoic acid (DPA) may be administered in an orally administrable composition comprising DPA. In some embodiments, the compositions comprise DPA in an amount between 1% and 99% relative to the total amount of fatty acids present in the composition, alternatively between 1% and 95%, alternatively between 1% and 90%, alternatively between 1% and 85%, alternatively between 1% and 80%, alternatively between 1% and 75%, alternatively between 1% and 70%, alternatively between 1% and 65%, alternatively between 1% and 60%, alternatively between 1% and 55%, alternatively between 1% and 50%, alternatively between 1% and 45%, alternatively between 1% and 40%, alternatively between 1% and 35%, alternatively between 1% and 30%, alternatively between 1% and 25%, alternatively between 1% and 20%, alternatively between 1% and 15%, alternatively between 1% and 10%, alternatively between 1% and 5%, alternatively between 2% and 99%, alternatively between 2% and 95%, alternatively between 2% and 90%, alternatively between 2% and 85%, alternatively between 2% and 80%, alternatively between 2% and 75%, alternatively between 2% and 70%, alternatively between 2% and 65%, alternatively between 2% and 60%, alternatively between 2% and 55%, alternatively between 2% and 50%, alternatively between 2% and 45%, alternatively between 2% and 40%, alternatively between 2% and 35%, alternatively between 2% and 30%, alternatively between 2% and 25%, alternatively between 2% and 20%, alternatively between 2% and 15%, alternatively between 2% and 10%, alternatively between 2% and 5%, alternatively between 3% and 99%, alternatively between 3% and 95%, alternatively between 3% and 90%, alternatively between 3% and 85%, alternatively between 3% and 80%, alternatively between 3% and 75%, alternatively between 3% and 70%, alternatively between 3% and 65%, alternatively between 3% and 60%, alternatively between 3% and 55%, alternatively between 3% and 50%, alternatively between 3% and 45%, alternatively between 3% and 40%, alternatively between 3% and 35%, alternatively between 3% and 30%, alternatively between 3% and 25%, alternatively between 3% and 20%, alternatively between 3% and 15%, alternatively between 3% and 10%, alternatively between 3% and 5%, alternatively between 4% and 99%, alternatively between 4% and 95%, alternatively between 4% and 90%, alternatively between 4% and 85%, alternatively between 4% and 80%, alternatively between 4% and 75%, alternatively between 4% and 70%, alternatively between 4% and 65%, alternatively between 4% and 60%, alternatively between 4% and 55%, alternatively between 4% and 50%, alternatively between 4% and 45%, alternatively between 4% and 40%, alternatively between 4% and 35%, alternatively between 4% and 30%, alternatively between 4% and 25%, alternatively between 4% and 20%, alternatively between 4% and 15%, alternatively between 4% and 10%, alternatively between 4% and 5%, alternatively between 5% and 99%, alternatively between 5% and 95%, alternatively between 5% and 90%, alternatively between 5% and 85%, alternatively between 5% and 80%, alternatively between 5% and 75%, alternatively between 5% and 70%, alternatively between 5% and 65%, alternatively between 5% and 60%, alternatively between 5% and 55%, alternatively between 5% and 50%, alternatively between 5% and 45%, alternatively between 5% and 40%, alternatively between 5% and 35%, alternatively between 5% and 30%, alternatively between 5% and 25%, alternatively between 5% and 20%, alternatively between 5% and 15%, alternatively between 5% and 12%, alternatively between 5% and 10%, alternatively between 6% and 99%, alternatively between 6% and 95%, alternatively between 6% and 90%, alternatively between 6% and 85%, alternatively between 6% and 80%, alternatively between 6% and 75%, alternatively between 6% and 70%, alternatively between 6% and 65%, alternatively between 6% and 60%, alternatively between 6% and 55%, alternatively between 6% and 50%, alternatively between 6% and 45%, alternatively between 6% and 40%, alternatively between 6% and 35%, alternatively between 6% and 30%, alternatively between 6% and 25%, alternatively between 6% and 20%, alternatively between 6% and 15%, alternatively between 6% and 12%, alternatively between 6% and 11%, alternatively between 6% and 10%, alternatively between 7% and 99%, alternatively between 7% and 95%, alternatively between 7% and 90%, alternatively between 7% and 85%, alternatively between 7% and 80%, alternatively between 7% and 75%, alternatively between 7% and 70%, alternatively between 7% and 65%, alternatively between 7% and 60%, alternatively between 7% and 55%, alternatively between 7% and 50%, alternatively between 7% and 45%, alternatively between 7% and 40%, alternatively between 7% and 35%, alternatively between 7% and 30%, alternatively between 7% and 25%, alternatively between 7% and 20%, alternatively between 7% and 15%, alternatively between 7% and 12%, alternatively between 7% and 11%, alternatively between 7% and 10%, alternatively between 8% and 99%, alternatively between 8% and 95%, alternatively between 8% and 90%, alternatively between 8% and 85%, alternatively between 8% and 80%, alternatively between 8% and 75%, alternatively between 8% and 70%, alternatively between 8% and 65%, alternatively between 8% and 60%, alternatively between 8% and 55%, alternatively between 8% and 50%, alternatively between 8% and 45%, alternatively between 8% and 40%, alternatively between 8% and 35%, alternatively between 8% and 30%, alternatively between 8% and 25%, alternatively between 8% and 20%, alternatively between 8% and 15%, alternatively between 8% and 12%, alternatively between 9% and 95%, alternatively between 9% and 90%, alternatively between 9% and 85%, alternatively between 9% and 80%, alternatively between 9% and 75%, alternatively between 9% and 70%, alternatively between 9% and 65%, alternatively between 9% and 60%, alternatively between 9% and 55%, alternatively between 9% and 50%, alternatively between 9% and 45%, alternatively between 9% and 40%, alternatively between 9% and 35%, alternatively between 9% and 30%, alternatively between 9% and 25%, alternatively between 9% and 20%, alternatively between 9% and 15%, alternatively between 9% and 12%, relative to the total amount of fatty acids present in the composition. In some embodiments, the compositions comprise DPA in an amount of at least about 45% of DPA. In some alternative embodiments, the composition comprises at least 45% or at least 50% or at least 55% or at least 60% or at least 65% or at least 70% or at least 75% or at least 80% or at least 85% or at least 90% or at least 95% of DPA. In some embodiments, the composition comprises at least 20 mg of DPA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DPA. In some embodiments, the composition comprises DPA in ester form or in free fatty acid form.

The methods of treatment provides a dose of at least 20 mg DPA-N3, alternatively at least 30 mg DPA-N3, alternatively at least 40 mg DPA-N3, alternatively at least 50 mg DPA-N3, alternatively at least 60 mg DPA-N3 per day, alternatively at least 70 mg DPA-N3 per day, alternatively at least 75 mg DPA-N3 per day, alternatively at least 80 mg DPA-N3 per day, alternatively at least 90 mg DPA-N3 per day, alternatively at least 100 mg DPA-N3 per day, alternatively at least 120 mg DPA-N3 per day, alternatively at least 150 mg DPA-N3 per day, alternatively at least 160 mg DPA-N3 per day, alternatively at least 180 mg DPA-N3 per day, alternatively at least 200 mg DPA-N3 per day, alternatively at least 250 mg DPA-N3 per day, alternatively at least 300 mg DPA-N3 per day, alternatively at least 350 mg DPA-N3 per day, alternatively at least 400 mg DPA-N3 per day, alternatively at least 500 mg DPA-N3 per day, alternatively at least 600 mg DPA-N3 per day, alternatively at least 800 mg DPA-N3 or its glycerol or ethyl esters per day.

In some embodiments, the method of treatment provides a daily dose of at least about at least 20 mg of DPA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DPA. In some embodiments, the method of treatment provides a daily dose of at least about 1,000 mg DPA-N3 per day, alternatively at least about 1,500 mg DPA-N3 per day, alternatively at least about 2,000 mg DPA-N3 per day, alternatively at least about 2,500 mg DPA-N3 per day, alternatively at least about 3,000 mg DPA-N3 per day, alternatively at least about 3,500 mg DPA-N3 per day, alternatively at least about 3,750 mg DPA-N3 per day, alternatively at least about 4,000 mg DPA-N3 per day, alternatively at least about 4,250 mg DPA-N3 per day.

In some embodiments, the method of treatment provides a dose of at least about 1 mg/kg of DPA-N3 per day, alternatively about 2 mg/kg of DPA-N3 per day, alternatively about 3 mg/kg of DPA-N3 per day, alternatively about 4 mg/kg of DPA-N3 per day, alternatively about 6 mg/kg of DPA-N3 per day, alternatively about 8 mg/kg of DPA-N3 per day, alternatively about 10 mg/kg of DPA-N3 per day, alternatively about 20 mg/kg of DPA-N3 per day, alternatively about 30 mg/kg of DPA-N3 per day, and alternatively about 40 mg/kg alternatively about 50 mg/kg of DPA-N3 per day, alternatively about 75 mg/kg of DPA-N3 per day, and alternatively about 100 mg/kg.

In some embodiments, the method of treatment provides a dose of about 0 mg/kg to 1 mg/kg of DPA-N3 per day, alternatively about 1 mg/kg to 2 mg/kg of DPA-N3 per day, alternatively about 2 mg/kg to 3 mg/kg of DPA-N3 per day, alternatively about 3 mg/kg to 4 mg/kg of DPA-N3 per day, alternatively about 4 mg/kg to 6 mg/kg of DPA-N3 per day, alternatively about 6 mg/kg to 8 mg/kg of DPA-N3 per day, alternatively about 8 mg/kg to 10 mg/kg of DPA-N3 per day, alternatively about 10 mg/kg to 20 mg/kg of DPA-N3 per day, alternatively about 20 mg/kg to 30 mg/kg of DPA-N3 per day, and alternatively about 30 mg/kg to 40 mg/kg alternatively about 40 mg/kg to 50 mg/kg of DPA-N3 per day, alternatively about 50 mg/kg to 75 mg/kg of DPA-N3 per day, and alternatively about 75 mg/kg to 100 mg/kg.

The docosahexaenoic acid (DHA) may be administered in an orally administrable composition comprising DHA. In some embodiments, the compositions comprise DHA in an amount between 1% and 99% relative to the total amount of fatty acids present in the composition, alternatively between 1% and 95%, alternatively between 1% and 90%, alternatively between 1% and 85%, alternatively between 1% and 80%, alternatively between 1% and 75%, alternatively between 1% and 70%, alternatively between 1% and 65%, alternatively between 1% and 60%, alternatively between 1% and 55%, alternatively between 1% and 50%, alternatively between 1% and 45%, alternatively between 1% and 40%, alternatively between 1% and 35%, alternatively between 1% and 30%, alternatively between 1% and 25%, alternatively between 1% and 20%, alternatively between 1% and 15%, alternatively between 1% and 10%, alternatively between 1% and 5%, alternatively between 2% and 99%, alternatively between 2% and 95%, alternatively between 2% and 90%, alternatively between 2% and 85%, alternatively between 2% and 80%, alternatively between 2% and 75%, alternatively between 2% and 70%, alternatively between 2% and 65%, alternatively between 2% and 60%, alternatively between 2% and 55%, alternatively between 2% and 50%, alternatively between 2% and 45%, alternatively between 2% and 40%, alternatively between 2% and 35%, alternatively between 2% and 30%, alternatively between 2% and 25%, alternatively between 2% and 20%, alternatively between 2% and 15%, alternatively between 2% and 10%, alternatively between 2% and 5%, alternatively between 3% and 99%, alternatively between 3% and 95%, alternatively between 3% and 90%, alternatively between 3% and 85%, alternatively between 3% and 80%, alternatively between 3% and 75%, alternatively between 3% and 70%, alternatively between 3% and 65%, alternatively between 3% and 60%, alternatively between 3% and 55%, alternatively between 3% and 50%, alternatively between 3% and 45%, alternatively between 3% and 40%, alternatively between 3% and 35%, alternatively between 3% and 30%, alternatively between 3% and 25%, alternatively between 3% and 20%, alternatively between 3% and 15%, alternatively between 3% and 10%, alternatively between 3% and 5%, alternatively between 4% and 99%, alternatively between 4% and 95%, alternatively between 4% and 90%, alternatively between 4% and 85%, alternatively between 4% and 80%, alternatively between 4% and 75%, alternatively between 4% and 70%, alternatively between 4% and 65%, alternatively between 4% and 60%, alternatively between 4% and 55%, alternatively between 4% and 50%, alternatively between 4% and 45%, alternatively between 4% and 40%, alternatively between 4% and 35%, alternatively between 4% and 30%, alternatively between 4% and 25%, alternatively between 4% and 20%, alternatively between 4% and 15%, alternatively between 4% and 10%, alternatively between 4% and 5%, alternatively between 5% and 99%, alternatively between 5% and 95%, alternatively between 5% and 90%, alternatively between 5% and 85%, alternatively between 5% and 80%, alternatively between 5% and 75%, alternatively between 5% and 70%, alternatively between 5% and 65%, alternatively between 5% and 60%, alternatively between 5% and 55%, alternatively between 5% and 50%, alternatively between 5% and 45%, alternatively between 5% and 40%, alternatively between 5% and 35%, alternatively between 5% and 30%, alternatively between 5% and 25%, alternatively between 5% and 20%, alternatively between 5% and 15%, alternatively between 5% and 12%, alternatively between 5% and 10%, alternatively between 6% and 99%, alternatively between 6% and 95%, alternatively between 6% and 90%, alternatively between 6% and 85%, alternatively between 6% and 80%, alternatively between 6% and 75%, alternatively between 6% and 70%, alternatively between 6% and 65%, alternatively between 6% and 60%, alternatively between 6% and 55%, alternatively between 6% and 50%, alternatively between 6% and 45%, alternatively between 6% and 40%, alternatively between 6% and 35%, alternatively between 6% and 30%, alternatively between 6% and 25%, alternatively between 6% and 20%, alternatively between 6% and 15%, alternatively between 6% and 12%, alternatively between 6% and 11%, alternatively between 6% and 10%, alternatively between 7% and 99%, alternatively between 7% and 95%, alternatively between 7% and 90%, alternatively between 7% and 85%, alternatively between 7% and 80%, alternatively between 7% and 75%, alternatively between 7% and 70%, alternatively between 7% and 65%, alternatively between 7% and 60%, alternatively between 7% and 55%, alternatively between 7% and 50%, alternatively between 7% and 45%, alternatively between 7% and 40%, alternatively between 7% and 35%, alternatively between 7% and 30%, alternatively between 7% and 25%, alternatively between 7% and 20%, alternatively between 7% and 15%, alternatively between 7% and 12%, alternatively between 7% and 11%, alternatively between 7% and 10%, alternatively between 8% and 99%, alternatively between 8% and 95%, alternatively between 8% and 90%, alternatively between 8% and 85%, alternatively between 8% and 80%, alternatively between 8% and 75%, alternatively between 8% and 70%, alternatively between 8% and 65%, alternatively between 8% and 60%, alternatively between 8% and 55%, alternatively between 8% and 50%, alternatively between 8% and 45%, alternatively between 8% and 40%, alternatively between 8% and 35%, alternatively between 8% and 30%, alternatively between 8% and 25%, alternatively between 8% and 20%, alternatively between 8% and 15%, alternatively between 8% and 12%, alternatively between 9% and 95%, alternatively between 9% and 90%, alternatively between 9% and 85%, alternatively between 9% and 80%, alternatively between 9% and 75%, alternatively between 9% and 70%, alternatively between 9% and 65%, alternatively between 9% and 60%, alternatively between 9% and 55%, alternatively between 9% and 50%, alternatively between 9% and 45%, alternatively between 9% and 40%, alternatively between 9% and 35%, alternatively between 9% and 30%, alternatively between 9% and 25%, alternatively between 9% and 20%, alternatively between 9% and 15%, alternatively between 9% and 12%, relative to the total amount of fatty acids present in the composition. In some embodiments, the compositions comprise DHA in an amount of at least about 45% of DHA. In some alternative embodiments, the composition comprises at least 45% or at least 50% or at least 55% or at least 60% or at least 65% or at least 70% or at least 75% or at least 80% or at least 85% or at least 90% or at least 95% of DHA. In some embodiments, the composition comprises at least 20 mg of DHA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DHA. In some embodiments, the composition comprises DHA in ester form or in free fatty acid form.

The methods of treatment provides a dose of at least 20 mg DHA-N3, alternatively at least 30 mg DHA-N3, alternatively at least 40 mg DHA-N3, alternatively at least 50 mg DHA-N3, alternatively at least 60 mg DHA-N3 per day, alternatively at least 70 mg DHA-N3 per day, alternatively at least 75 mg DHA-N3 per day, alternatively at least 80 mg DHA-N3 per day, alternatively at least 90 mg DHA-N3 per day, alternatively at least 100 mg DHA-N3 per day, alternatively at least 120 mg DHA-N3 per day, alternatively at least 150 mg DHA-N3 per day, alternatively at least 160 mg DHA-N3 per day, alternatively at least 180 mg DHA-N3 per day, alternatively at least 200 mg DHA-N3 per day, alternatively at least 250 mg DHA-N3 per day, alternatively at least 300 mg DHA-N3 per day, alternatively at least 350 mg DHA-N3 per day, alternatively at least 400 mg DHA-N3 per day, alternatively at least 500 mg DHA-N3 per day, alternatively at least 600 mg DHA-N3 per day, alternatively at least 800 mg DHA-N3 or its glycerol or ethyl esters per day.

In some embodiments, the method of treatment provides a daily dose of at least about at least 20 mg of DHA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DHA. In some embodiments, the method of treatment provides a daily dose of at least about 1,000 mg DHA-N3 per day, alternatively at least about 1,500 mg DHA-N3 per day, alternatively at least about 2,000 mg DHA-N3 per day, alternatively at least about 2,500 mg DHA-N3 per day, alternatively at least about 3,000 mg DHA-N3 per day, alternatively at least about 3,500 mg DHA-N3 per day, alternatively at least about 3,750 mg DHA-N3 per day, alternatively at least about 4,000 mg DHA-N3 per day, alternatively at least about 4,250 mg DHA-N3 per day.

In some embodiments, the method of treatment provides a dose of at least about 1 mg/kg of DHA-N3 per day, alternatively about 2 mg/kg of DHA-N3 per day, alternatively about 3 mg/kg of DHA-N3 per day, alternatively about 4 mg/kg of DHA-N3 per day, alternatively about 6 mg/kg of DHA-N3 per day, alternatively about 8 mg/kg of DHA-N3 per day, alternatively about 10 mg/kg of DHA-N3 per day, alternatively about 20 mg/kg of DHA-N3 per day, alternatively about 30 mg/kg of DHA-N3 per day, and alternatively about 40 mg/kg alternatively about 50 mg/kg of DHA-N3 per day, alternatively about 75 mg/kg of DHA-N3 per day, and alternatively about 100 mg/kg.

In some embodiments, the method of treatment provides a dose of about 0 mg/kg to 1 mg/kg of DHA-N3 per day, alternatively about 1 mg/kg to 2 mg/kg of DHA-N3 per day, alternatively about 2 mg/kg to 3 mg/kg of DHA-N3 per day, alternatively about 3 mg/kg to 4 mg/kg of DHA-N3 per day, alternatively about 4 mg/kg to 6 mg/kg of DHA-N3 per day, alternatively about 6 mg/kg to 8 mg/kg of DHA-N3 per day, alternatively about 8 mg/kg to 10 mg/kg of DHA-N3 per day, alternatively about 10 mg/kg to 20 mg/kg of DHA-N3 per day, alternatively about 20 mg/kg to 30 mg/kg of DHA-N3 per day, and alternatively about 30 mg/kg to 40 mg/kg alternatively about 40 mg/kg to 50 mg/kg of DHA-N3 per day, alternatively about 50 mg/kg to 75 mg/kg of DHA-N3 per day, and alternatively about 75 mg/kg to 100 mg/kg.

The present invention also provides an administrable composition comprising fatty acids, wherein at least 50% by weight of the fatty acids comprise omega-3-fatty acids, salts, esters, or derivatives thereof, wherein the omega-3 fatty acids comprise eicosapentaenoic acid (EPA; C20:5-n3), docosapentaenoic acid (DPA; C22:5-n3), and docosahexaenoic acid (DHA; C22:6-n3), wherein the ratio of DHA to EPA (DHA:DPA) is less than 1:20, and wherein the ratio of DHA to DPA (DHA:DPA) is less than 2:1.

In some embodiments, the compositions of the present invention comprise at least 50% omega-3 fatty acids, alternatively at least 55%, alternatively at least 60%, alternatively at least 65%, alternatively at least 70%, alternatively at least 75%, alternatively at least 80%, alternatively at least 85%, alternatively at least 95%, most preferably at least 90% omega-3 fatty acids of the total amount of fatty acids.

In other embodiments, EPA and DPA are jointly present in the compositions of the present invention at between 55% and 100% of total fatty acids, alternatively between 60% and 100%, alternatively between 65% and 100%, alternatively between 70% and 100%, alternatively between 75% and 100%, alternatively between 80% and 100%, alternatively between 85% and 95%, alternatively between 85% and 97%, alternatively between 88% and 95%, alternatively between 88% and 97%, alternatively between 90% and 95%, alternatively between 90% and 97% of the total amount of fatty acids.

In other embodiments, EPA and DHA are jointly present in the compositions of the present invention at between 55% and 100% of total fatty acids, alternatively between 60% and 100%, alternatively between 65% and 100%, alternatively between 70% and 100%, alternatively between 75% and 100%, alternatively between 80% and 100%, alternatively between 85% and 95%, alternatively between 85% and 97%, alternatively between 88% and 95%, alternatively between 88% and 97%, alternatively between 90% and 95%, alternatively between 90% and 97% of the total amount of fatty acids.

The fatty acids, such as EPA, DHA, and DPA, may be present in free fatty acid form, or as a salt, ester, or derivative. The fatty acids are preferably composed as a triglyceride, an ester (such as an ethyl ester) or free fatty acid. Other forms of the fatty acids which may be useful include salts, esters of any type, amides, mono-, di- or triglycerides, phospholipids or any other form which can lead to metabolization of the fatty acids (such as EPA, DHA, and/or DPA), or the incorporation of the fatty acids (such as EPA and/or DPA) into body fluids, tissues or organs.

In some embodiments, the compositions of the present invention comprise at least 0.01% HPA of total fatty acids in the composition, alternatively at least 0.05% HPA, alternatively at least 0.10% HPA, alternatively at least 0.15% HPA, alternatively at least 0.2% HPA, alternatively at least 0.3% HPA, alternatively at least 0.4% HPA, alternatively at least 0.5% HPA, alternatively at least 0.75% HPA, alternatively at least 1% HPA, alternatively at least 1.5% HPA, alternatively at least 2% HPA, alternatively at least 2.5% HPA, alternatively at least 3% HPA, alternatively at least 3.5% HPA, alternatively at least 4% HPA, alternatively at least 4.5% HPA, alternatively at least 5% HPA, alternatively at least 6% HPA, alternatively at least 7% HPA, alternatively the compositions of the present invention comprise at least 9% HPA of total fatty acids in the composition.

In some embodiments, the compositions of the present invention comprise no more than 20% HPA of total fatty acids in the composition, alternatively no more than 15% HPA, alternatively no more than 12% HPA, alternatively no more than 10% HPA, alternatively no more than 8% HPA, alternatively no more than 7% HPA, alternatively no more than 6% HPA, alternatively no more than 5% HPA, alternatively no more than 4% HPA, alternatively no more than 3% HPA, alternatively no more than 2% HPA, alternatively no more than 1.5% HPA, alternatively the compositions of the present invention comprise at least 1% HPA of total fatty acids in the composition. In some embodiments, the compositions of the present invention comprise 1% to 20% of the total fatty acids in the composition.

In the embodiments of the present invention, the compositions comprise EPA and DPA in an EPA:DPA ratio between 99:1 and 1:99 EPA:DPA, alternatively between 90:1 and 1:90, alternatively between 60:1 and 1:60, alternatively between 60:1 and 1:20, alternatively between 60:1 and 1:4, alternatively between 40:1 and 1:20, alternatively between 30:1 and 1:20, alternatively between 30:1 and 1.10, alternatively between 30:1 and 1:5, alternatively between 40:1 and 1:4, alternatively between 30:1 and 1:4, alternatively between 30:1 and 1:2, alternatively between 30:1 and 1:1, alternatively between 30:1 and 2:1, alternatively between 30:1 and 5:1, alternatively between 20:1 and 1:20, alternatively between 20:1 and 1:10, alternatively between 20:1 and 1:5, alternatively between 20:1 and 1:2, alternatively between 20:1 and 1:1, alternatively between 20.1 and 2:1, alternatively between 20:1 and 5:1, alternatively between 20:1 and 10:1, alternatively between 20:1 and 10:1, alternatively between 30:1 and 10:1, alternatively between 60:1 and 10:1, alternatively comprise EPA and DPA in an EPA:DPA ratio between 40:1 and 10:1. In some embodiments, the ratio of EPA:DPA is greater than 1:1, preferably greater than 2:1, and more preferably greater than 5:1. In some embodiments, the ratio of EPA:DPA is 1:1 to 25:1, preferably 5:1 to 20:1, more preferably 8:1 to 15:1, even more preferably 9:1 to 13:1, even more most preferably about 10:1 to 11:1, and most preferably about 10:1.

In the embodiments of the present invention, the compositions comprise EPA and DHA in an EPA:DHA ratio between 99:1 and 1:99 EPA:DHA, alternatively between 90:1 and 1:90, alternatively between 60:1 and 1:60, alternatively between 60:1 and 1:20, alternatively between 60:1 and 1:4, alternatively between 40:1 and 1:20, alternatively between 30:1 and 1:20, alternatively between 30:1 and 1:10, alternatively between 30:1 and 1:5, alternatively between 40:1 and 1:4, alternatively between 30:1 and 1:4, alternatively between 30:1 and 1:2, alternatively between 30:1 and 1:1, alternatively between 30:1 and 2:1, alternatively between 30:1 and 5:1, alternatively between 20:1 and 1:20, alternatively between 20:1 and 1:10, alternatively between 20:1 and 1:5, alternatively between 20:1 and 1:2, alternatively between 20:1 and 1:1, alternatively between 20:1 and 2:1, alternatively between 20:1 and 5:1, alternatively between 20:1 and 10:1, alternatively between 20:1 and 10:1, alternatively between 30:1 and 10:1, alternatively between 60:1 and 10:1, alternatively comprise EPA and DHA in an EPA:DHA ratio between 40:1 and 10:1. In some embodiments, the ratio of EPA:DHA is greater than 1:1, preferably greater than 2:1, and more preferably greater than 5.1. In some embodiments, the ratio of EPA:DHA is 1:1 to 25:1, preferably 5:1 to 20:1, more preferably 8:1 to 15:1, even more preferably 9:1 to 13:1, even more most preferably about 10:1 to 11:1, and most preferably about 10:1.

In some embodiments, a relatively small amount of DHA relative to the total amount of fatty acids present in the composition is present. In some embodiments, the compositions of the present invention comprise no more than 20% DHA, alternatively no more than 15% DHA, alternatively no more than 12% DHA, alternatively no more than 10% DHA, alternatively no more than 8% DHA, alternatively no more than 7% DHA, alternatively no more than 6% DHA, alternatively no more than 5% DHA, alternatively no more than 4% DHA, alternatively no more than 3% DHA, alternatively no more than 2% DHA, alternatively no more than 1% DHA relative to the total amount of fatty acids present in the composition.

In some embodiments, the ratio of DPA:HPA is about 250:1 to 1:1, alternatively 200:1 to 2:1, alternatively 150:1 to 3:1, alternatively 100:1 to 4:1, alternatively 50:1 to 5:1, alternatively 25:1 to 6:1, and alternatively 10:1 to 7:1. In some preferred embodiments, the ratio of DPA:HPA is about 8:1. In some embodiments, the ratio of DPA:HPA is about 3:0.

In some embodiments, the ratio of DHA:DPA is about 250:1 to 1:1, alternatively 200:1 to 2:1, alternatively 150:1 to 3:1, alternatively 100:1 to 4:1, alternatively 50:1 to 5:1, alternatively 25:1 to 6:1, and alternatively 10:1 to 7:1. In some preferred embodiments, the ratio of DHA:DPA is about 8:1. In some embodiments, the ratio of DHA:DPA is about 3:0.

In yet other embodiments, the compositions of the present invention comprise no more than 10% omega-6 fatty acids relative to the total amount of fatty acids, alternatively no more than 9%, alternatively no more than 8%, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% omega-6 fatty acids versus the total amount of fatty acids comprised by the compositions of the present invention.

Omega-6 fatty acids include, but are not limited to: linoleic acid (LA; C18:2-n6); gamma-linoleic acid (GLA; C18:3-n6); eicosadienoic acid (C20:2-n6); dihomo-gamma-linoleic acid (DGLA; C20:3-n6); arachiconic acid (ARA; C20:4-n6); and omega-6 docosapentaenoic acid (DPA; C22:5-n6).

In further embodiments, the compositions of the present invention comprise no more than 10% omega-6 fatty acids relative to the total amount of omega-3 fatty acids plus omega-6 fatty acids, alternatively no more than 9%, alternatively no more than 8%, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% omega-6 fatty acids versus the total amount of omega-3 fatty acids plus omega-6 fatty acids comprised by the compositions of the present invention.

In yet other embodiments, the compositions of the present invention comprise no more than 8% arachidonic acid (ARA; C20:4-n6) relative to the total amount of omega-3 fatty acids plus omega-6 fatty acids, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% arachidonic acid (ARA; C20:4-n6) versus the total amount of omega-3 fatty acids plus omega-6 fatty acids comprised by the compositions of the present invention.

In some embodiments, a relatively small amount of omega-3 fatty acids in aggregate other than EPA. ETA, HPA and DPA (alternatively indicated as non-EPA, non-ETA, non-HPA and non-DPA omega-3 fatty acids in aggregate) relative to the total amount of fatty acids present in the composition is present. In some embodiments, the compositions of the present invention comprise no more than 20% non-EPA, non-ETA, non-HPA and non-DPA omega-3 fatty acids, alternatively no more than 15% non-EPA, non-ETA, non-HPA and non-DPA omega-3 fatty acids, alternatively no more than 12% non-EPA, non-ETA, non-HPA and non-DPA omega-3 fatty acids, alternatively no more than 10% non-EPA, non-ETA, non-HPA and non-DPA omega-3 fatty acids, alternatively no more than 8% non-EPA, non-ETA, non-HPA and non-DPA omega-3 fatty acids, alternatively no more than 7% non-EPA, non-ETA, non-HPA and non-DPA omega-3 fatty acids, alternatively no more than 6% non-EPA, non-ETA, non-HPA and non-DPA omega-3 fatty acids, alternatively no more than 5% non-EPA, non-ETA, non-HPA and non-DPA omega-3 fatty acids, alternatively no more than 4% non-EPA, non-ETA, non-HPA and non-DPA omega-3 fatty acids, alternatively no more than 3% non-EPA, non-ETA, non-HPA and non-DPA omega-3 fatty acids, alternatively no more than 2% non-EPA, non-ETA, non-HPA and non-DPA omega-3 fatty acids, alternatively no more than 1% non-EPA, non-ETA, non-HPA and non-DPA omega-3 fatty acids in aggregate relative to the total amount of fatty acids present in the composition.

In some embodiments, a relatively small amount of the sum of ALA, SDA and DHA relative to the total amount of fatty acids present in the composition is present, while at the same time large amounts of the sum of EPA, DPA-n3, HPA and ETA are present. In some embodiments, the compositions of the present invention comprise no more than 20% of the sum of ALA, SDA and DHA, alternatively no more than 15% of the sum of ALA, SDA and DHA, alternatively no more than 12% of the sum of ALA, SDA and DHA, alternatively no more than 10% of the sum of ALA, SDA and DHA, alternatively no more than 8% of the sum of ALA, SDA and DHA, alternatively no more than 7% of the sum of ALA, SDA and DHA, alternatively no more than 6% of the sum of ALA, SDA and DHA, alternatively no more than 5% of the sum of ALA, SDA and DHA, alternatively no more than 4% of the sum of ALA, SDA and DHA, alternatively no more than 3% of the sum of ALA, SDA and DHA, alternatively no more than 2% of the sum of ALA, SDA and DHA, alternatively no more than 1% of the sum of ALA, SDA and DHA relative to the total amount of fatty acids present in the composition, while at the same time contain more than 40% the sum of EPA, DPAn-3, HPA and ETA, alternatively more than 50% the sum of EPA, DPAn-3, HPA and ETA, alternatively more than 60% the sum of EPA, DPAn-3, HPA and ETA, alternatively more than 70% the sum of EPA, DPAn-3, HPA and ETA, alternatively more than 75% the sum of EPA, DPAn-3, HPA and ETA, alternatively more than 80% the sum of EPA, DPAn-3, HPA and ETA, alternatively more than 85% the sum of EPA, DPAn-3, HPA and ETA, alternatively more than 90% the sum of EPA, DPAn-3, HPA and ETA, alternatively more than 95% the sum of EPA, DPAn-3, HPA and ETA, alternatively between 80% and 98% the sum of EPA, DPAn-3, HPA and ETA, alternatively between 80% and 96% the sum of EPA, DPAn-3, HPA and ETA, alternatively between 85% and 98% the sum of EPA, DPAn-3, HPA and ETA, alternatively between 85% and 96% the sum of EPA. DPAn-3, HPA and ETA, alternatively between 90% and 98% the sum of EPA, DPAn-3, HPA and ETA, alternatively between 90% and 97% the sum of EPA. DPAn-3, HPA and ETA, alternatively between 90% and 96% the sum of EPA, DPAn-3, HPA and ETA, alternatively between 90% and 95% the sum of EPA, DPAn-3, HPA and ETA, relative to the total amount of fatty acids present in the composition is present.

In further embodiments, the compositions of the present invention comprise no more than 8% arachidonic acid (ARA; C20:4-n6) relative to the total amount of fatty acids, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% arachidonic acid (ARA; C20:4-n6) relative the total amount of fatty acids comprised by the compositions of the present invention.

In some embodiments, the composition of the present invention further comprises TPA at concentration of at least 0.05%. In some embodiments, the TPA concentration is about 0.01% to about 5%, alternatively about 0.05% to about 2%, alternatively about 0.1% to about 1%, alternatively about 0.2% to about 0.8%, alternatively about 0.4% to about 0.6%, alternatively about 0.5%.

In other embodiments, the compositions of the present invention comprise no more than 2.5% arachidonic acid (ARA; C20:4-n6), no more than 0.4% omega-6-docosapentaenoic acid (DPA; C22:5-n6) and no more than 0.2% gamma-linoleic acid (GLA; C18:3-n6) relative the total amount of fatty acids comprised by the compositions of the present invention.

Further embodiments provide fatty acid compositions comprising no more than 2.5% arachidonic acid (ARA; C20:4-n6), no more than 0.3% omega-6 docosapentaenoic acid (DPA; C22:5-n6) and no more than 0.1% gamma-linoleic acid (GLA; C183-n6) relative the total amount of fatty acids comprised by the compositions of the present invention.

In yet other embodiments, the active ingredient of the formulations of the present invention consists essentially wholly of the DPA and DHA or precursors thereof (ethyl ester, triglyceride, or any other pharmaceutically acceptable salt or derivative thereof). In that case, no large amounts (preferably less than 15%, alternatively less than 12%, alternatively less than 10%, alternatively less than 9%, alternatively less than 8%, alternatively less than 7%, alternatively less than 6%, alternatively less than 5%, alternatively less than 4%, alternatively less than 3%, alternatively less than 2%, alternatively less than 1%, alternatively less than 0.5%, alternatively less than 0.25%) of any other fatty acids are present.

The fatty acid percentage is determined on a weight/weight, mol/mol, or chromatography area percent basis relative to all fatty acids present in the composition as determined by methods such as disclosed in the European Pharmacopeia monograph for omega-3 fatty acid concentrates, European Pharmacopeia monograph for omega-3-acid ethyl esters 90%, or European Pharmacopeia monograph method 2.4.29, USP monograph for fish oil dietary supplements, USP 35 omega-3-acid ethyl esters (LOVAZA®) monograph, or any essentially equivalent methods (whether by gas chromatography, HPLC, FPLC or any other chromatographic method).

In some embodiments, the fatty acid percentage is determined not as a percentage of all fatty acids present in the composition but as a specific type of fatty acid ethyl esters as percentage of all fatty acid ethyl esters present in the composition, thus excluding from the fatty acid percentage determination such fatty acids present as, for instance: free fatty acids: mono-, di-, and tri-glycerides; or fatty acids present in phospholipids (such as phosphatidylserine or phosphatidylcholine) or polysorbates (such as Tween 80, Tween 20, or polysorbate 40).

In other embodiments, the fatty acid percentage is determined not as a percentage of all fatty acids present in the composition but as a specific type of free fatty acid as percentage of all free fatty acids present in the composition, thus excluding from the fatty acid percentage determination such fatty acids present as, for instance: fatty acid ethyl esters; mono-, di-, and tri-glycerides; or fatty acids present in phospholipids (such as phosphatidylserine or phosphatidylcholine) or polysorbates (such as Tween 80, Tween 20, or polysorbate 40).

In yet other embodiments, the fatty acid percentage is determined not as a percentage of all fatty acids present in the composition but as a specific type of glycerol fatty acid ester as percentage of all glycerol fatty acid esters present in the composition, thus excluding from the fatty acid percentage determination such fatty acids present as, for instance: fatty acid ethyl esters; free fatty acids; or fatty acids present in phospholipids (such as phosphatidylserine or phosphatidylcholine) or polysorbates (such as Tween 80, Tween 20, or polysorbate 40).

In further embodiments, the fatty acid percentage is determined not as a percentage of all fatty acids present in the composition but as di- or tri-fatty acid esters with glycerol as percentage of all glycerol di- and tri-fatty acid esters present in the composition, thus excluding from the fatty acid percentage determination such fatty acids present as, for instance: glycerol-mono-fatty acid esters; fatty acid ethyl esters; free fatty acids; or fatty acids present in phospholipids (such as phosphatidylserine or phosphatidylcholine) or polysorbates (such as Tween 80, Tween 20, or polysorbate 40).

In yet other embodiments, the fatty acid percentage is determined not as a percentage of all fatty acids present in the composition but as a tri-fatty acid esters with glycerol as percentage of all glycerol tri-fatty acid esters present in the composition, thus excluding from the fatty acid percentage determination such fatty acids present as, for instance: mono- and di-fatty acid esters of glycerol; fatty acid ethyl esters; free fatty acids; or fatty acids present in phospholipids (such as phosphatidylserine or phosphatidylcholine) or polysorbates (such as Tween 80, Tween 20, or polysorbate 40).

The EPA, HPA, DPA, DHA, or omega-3-pentaenoic acids may be derived from any appropriate source including plant seed oils, microbial oils from algae or fungal or marine oils from fish or other marine animals. Certain species are a particular good source of oils containing DPA, for example seal oil. They may be used in the form of the natural oil, if that oil meets the required purity requirements of the present invention, or may be purified to give products containing the fatty acid composition of the present invention.

The compositions of the present invention may be produced through a range of the methods. Such methods may include: distillation, including short path distillation; urea precipitation; enzymatic conversion concentration; conventional chromatography; HPLC/FPLC; supercritical carbondioxide extraction; supercritical carbondioxide chromatography; simulated moving bed chromatography; supercritical carbondioxide simulated moving bed chromatography; or chemical conversion methods such as iodolactonization. Such methods are generally known to those skilled in the art of purifying and isolating omega-3 fatty acids.

Typically, the omega-3 fatty acid concentration/purification process is initiated by esterifying the fatty acids comprised by the marine oil raw material (such as crude fish oil) with ethanol (to form fatty acid ethyl esters) in order to separate omega-3 fatty acids from other fatty acids covalently bound together in the natural triglyceride molecules of the source oil. Subsequently, the material may be distilled once or several times to achieve omega-3-acid ethyl ester concentrations above 60%-70%. Alternatively, enzymatic concentration, urea precipitation or supercritical extraction may be used alone or in conjunction with distillation to reach omega-3 levels above 70%-90%. In order to prepare a highly pure concentrate of a single omega-3 fatty acid, methods such as chromatography, supercritical chromatography, simulated moving bed chromatography, supercritical simulated moving bed chromatography, or chemical conversion methods such as iodolactolization are typically most practical to reach levels above 50%, alternatively above 60%, alternatively above 70%, alternatively above 80%, alternatively above 90%, alternatively above 95%, of a single omega-3 fatty acid such as ETA, EPA, HPA, DPA, TPA, or DHA.

Those skilled in the art will be able to design processes suited to prepare a certain omega-3 fatty acid composition as desired, based on the methods described above. Such processes are flexible enough to affect the relative proportions between the long chain C18, C20, C21 and C22 fatty acids which occur naturally in available fish oil raw materials and other marine oils. It provides not only for the concentration of the individual omega-3 fatty acids, but the ratio between them will remain within a pattern of variation caused by variations in nature. However, suitable methods compensate for sometimes extreme variations which may occur naturally. Thus, for those skilled in the art, it will be possible to make a product with a constant and predetermined composition.

EPA is relatively abundant in fish oils or other marine oils and can be relatively easy obtained through the application of concentration and purification technologies from such fish or marine oils. DPA and HPA are present at much lower concentrations. In order to prepare the compositions of the present invention. DPA or HPA may be concentrated and purified from fish or other marine oils according to the methods referred to above, either alone or DPA combined with EPA and/or HPA. Alternatively, the DPA or HPA may be chemically prepared from a high purity EPA concentrate by elongation of the EPA fatty-acid chain with two or one hydrogen-saturated carbons (C2-elongation or C1-elongation) on the carboxyl side of the molecule (for instance with a method similar to or alternate methods with equivalent results such as described by Kuklev D V and Smith W L in Chem Phys Lipids, 2006; 144(2): 172-177). In another alternative approach, a high purity EPA concentrate may be partially converted to DPA (or HPA) using a method for C2-elongation (or C1-elongation) of EPA similar to those described above, thus directly yielding compositions of the present invention or intermediates therefore.

Once the oils containing one or more of the desired fatty acids have been obtained, and purified as necessary, these oils may be blended to give the desirable relative amounts of EPA, DPA, HPA, DHA, TPA, other omega-3 fatty acids and omega-6 fatty acids to obtain the compositions of the present invention described in detail above.

Fish oils may also contain by-products and contaminants such as pesticides, chlorinated or brominated hydrocarbons, heavy metals, cholesterol and vitamins. During the production of the concentrate, the concentrations of these components are significantly reduced compared to untreated fish oils. Such reduction is inherent due to the nature of purification methods and their ability to concentrate of several or specific omega-3 fatty acids, thus removing other compounds.

In some embodiments, triglycerides comprising more than 60% of the omega-3 fatty acids in the composition, alternatively more than 70%, alternatively more than 80%, alternatively more than 90% of the omega-3 fatty acids in the composition, may be produced from ethyl esters and glycerol by well known, published, or alternative chemical synthetic or enzymatic procedures. Di-glycerides (either 1,2 di-glycerides, 1.3 diglycerides, mixtures thereof) comprising more than 60% of the omega-3 fatty acids in the composition, alternatively more than 70%, alternatively more than 80%, alternatively more than 90% of the omega-3 fatty acids in the composition, may be produced from ethyl esters and glycerol by well known, published, or alternative chemical synthetic or enzymatic procedures. Mono-glycerides comprising more than 60% of the omega-3 fatty acids in the composition, alternatively more than 70%, alternatively more than 80%, alternatively more than 90% of the omega-3 fatty acids in the composition, may be produced from ethyl esters and glycerol by well known, published, or alternative chemical synthetic or enzymatic procedures. The free acids may be produced from ethyl esters by well known hydrolization or saponification procedures. Methods for converting ethyl esters to triglycerides, free fatty acids, and other molecular forms comprising fatty acids, are generally known to those skilled in the art chemically or enzymatically converting omega-3 fatty acids from one form to another.

The compositions of the present invention may be used for the treatment of patients by administering an effective amount of such compositions to a subject in need thereof, such as a subject prone to or afflicted with a disease or condition or in need of treatment for a disease or condition. The compositions of the present invention may be used for the treatment of patients by administering an effective amount of such compositions to a subject in need thereof, such as a subject prone to or afflicted with a disease or condition or in need of treatment for a disease or condition. The present invention provides methods of treating, preventing, and reducing the symptoms, pathology or events associated with a disease or condition comprising administration of any of the compositions of the present invention. The present invention provides methods of treating, preventing, and reducing the symptoms, pathology or events associated with liver-related disorders or conditions. Examples of liver-related disorders or conditions include, but are not limited to the following: fatty liver, non-alcoholic steaohepatitis (NASH), non-alcoholic fatty liver disease (NAFLD), alcoholic steatohepatitis (ASH), hepatitis including but not limited to hepatitis C, HIV (human immunodeficiency virus) infection and any liver syndrome arising from the treatment of HIV, drug-induced fatty liver or sequalae (including but not limited to NAFLD, NASH), liver failure, liver transplantation, transplanted liver failure, and liver damage. The liver damage may include damage associated with afflictions of other organ systems, such as renal failure or disease and diabetes, and damage caused or induced by drugs. The methods may be also be useful in the treatment of conditions associated with abnormally elevated liver enzymes (such as ALT, AST and/or GGT).

The effectiveness of the compositions of the present invention may be attributed in part to its activity in modulating lipid metabolism, including that of the liver, from a state of production and accumulation to a state of decreased production and removal vis-à-vis oxidation. Also, the compositions may elicit anti-inflammatory effects through a variety of mechanisms that can decrease levels of pro-inflammatory or pro-fibrotic mediators, such as TNF-alpha or TGF-beta, and increase levels of anti-inflammatory mediators, including IL-10 and arginase. The compositions may also be effective in decreasing the levels of lipids, specifically stored triglycerides, and to decrease pro-inflammatory mediators associated with fibrotic mechanisms.

The present invention further provides a method of reducing expression of HMG-CoA reductase and/or reducing levels of HMG-CoA reductase in a subject. The present invention further provides a method of reducing the expression of proprotein convertase subtilisin/kexin type 9 (PCSK9) and/or reducing levels of PCSK9 in a subject. Expression or levels may be determined by typical molecular biology methodologies or biochemical assays, such as mRNA levels to determine gene expression, ELISA assays to determine protein levels; or other techniques know to those with skill in the art.

The present invention provides methods of treating, preventing, and reducing symptoms associated with a disease or condition comprising administration of a composition of the present invention. Exemplary diseases or conditions include, but are not limited to: hypertriglyceridemia (for example, by those skilled in the art typically established by assessing fasting triglyceride (TG) levels); hypertriglyceridemia with TG≧500 mg/dL (VHTG); hypertriglyceridemia with TG 200-499 mg/dL; hypertriglyceridemia with TG 200-499 mg/dL while on statin treatment (HTG); hypercholesterolemia; mixed dyslipidemia; coronary heart disease (CHD); vascular disease; atherosclerotic disease and related conditions; heart failure; cardiac arrhythmias; blood coagulatory conditions associated with cardiac arrhytmias; hypertension; coagulation related disorders, including post-surgical deep vein thrombosis or other high risk thrombosis conditions; nephropathy; kidney or urinary tract disease; retinopathy; cognitive, psychiatric, neurological and other CNS disorders, including but not limited to schizophrenia, depression, bipolar disorder and any form of dementia (including ischemic dementia and vascular dementia); autoimmune diseases; inflammatory diseases; asthma, COPD or other respiratory disease; dermatological disease; metabolic syndrome; diabetes or other forms of metabolic disease; liver diseases including fatty liver disease; diseases affecting the senses, including those affecting vision and hearing; diseases of the gastrointestinal tract; diseases of the male or female reproductive system or related secondary sexual organs; a cancer of any type, including lymphomas, myelomas and solid tumor cancers; any infections caused by a virus, bacterium, fungus, protozoa or other organism. The present invention also provides for the treatment and/or prevention of cardiac events and/or cardiovascular events and/or vascular events and/or symptoms. The present invention also provides for the reduction of number of such events, as well as a reduction or amelioration of symptoms associated with such events.

The compositions of the present invention may be useful for any subjects, including those with and without comorbidities. In some embodiments, the subjects to be treated with or administered the compositions of the present invention, are selected based on the presence of certain comorbidities. In some embodiments, treatment with the compositions of the present invention results in clinical improvement of such comorbidities. In some embodiments, administration with the compositions of the present invention may reduce the time necessary to achieve clinical improvement and/or attain treatment goals. In some embodiments, the administration of compositions can result in clinical improvements in clinical markers, such as levels of liver enzymes and biomarkers. Clinical improvement relating to liver conditions, such as NAFLD and/or NASH, can be assessed utilizing a multitude of methodologies known to those practiced in the art. Liver biopsy histopathology scoring for fibrosis and ultrasound measurements of liver stiffness can be used for clinical assessment, measurement in the baseline changes in liver enzymes and any additional biomarkers such as but not limited to the FIBROTEST® and/or the nonalcoholic fatty liver disease (NAFLD) activity score, which is based on a standardized grading system for steatosis (on a scale of 0 to 3), lobular inflammation (on a scale of 0 to 3), and hepatocellular ballooning (on a scale of 0 to 2), with higher scores indicating increasing severity of disease.

The compositions of the present invention described for use in NASH can be administered by a multitude of routes, including oral, intravenous, topical, rectal and direct injection to the site of action.

The compositions of the present invention may be co-administered with one or more other therapeutic agents. In some embodiments, clinical benefits resulting from the administration or treatment of subjects with the compositions of the present invention may be improved with concomitant use or in combination with other therapeutic agents. The co-administration may result in a synergistic, more potent and efficacious course of treatment. In addition, it may be possible to use lower doses of these agents to elicit the synergistic effect, which may reduce the number and severity of adverse effects that are associated when they are used alone. Examples of such concomitant or fixed combination treatments may include coadministration with one or more of the following: PPAR gamma agents (such as pioglitazone), mixed PPAR agents such as those affecting the alpha/delta receptors, bile-acid derivatives, vitamin E, pentoxyfiline, hepatitis virus anti-viral agents, immunosuppressants associated with organ transplant, anti-platelet agents, anti-hypertensive drugs, diuretics, anti-inflammatory agents, anti-fibrotic agents, anti-cell death agents, anti-cell necrotic agents, and biological agents such as but not limited to monoclonal antibodies, or soluble receptors.

Cardiovascular and/or cardiac events that may benefit from treatment with the compositions of the present invention may include, but are not limited to: myocardial infarction, ischemic cardiac attack, ischemic attack, acute angina, hospitalization due to acute angina, stroke, transient ischemic cerebral attack, cardiac revascularization, cardiac revascularization with stent placement, carotid artery revascularization, carotid artery revascularization with stent placement, peripheral artery revascularization, peripheral artery revascularization with stent placement, plaque rupture, death due to cardiovascular event, and hospitalization due to cardiovascular event. Cardiovascular and/or cardiac events may also include other events deemed to fall in such category by those skilled in the art.

In some embodiments, the improved profile of the compositions of the present invention may be demonstrated upon treatment of a subject by differentially altering the ration between blood platelets and fragments thereof (also known as platelet microparticles). Such fragments may be evaluated as a whole or examined and described as fragment sub-categories.

In other embodiments, the improved profile of the compositions of the present invention may be demonstrated upon treatment of a subject by differentially altering the surface charge of blood platelets and fragments thereof, either in resting state (non-activated platelets) or activated stage.

In further embodiments, treatment with compositions of the present invention improves the vascular healing process in response to atherogenic disease. Such healing may be demonstrated by reduced stenosis and/or restenosis over time, reduced or lesser increase in intima-media thickness (IMT) of the arterial wall, larger lumen size and/or larger vascular diameter at vascular sites with stenosis or clot built-up, as determined by either by intravascular ultrasound (IVUS), radiographic, radiologic, non-invasive ultrasound, tomography, magnetic resonance interference (MRI), or other acceptable methods. In other embodiments, such improved healing may be demonstrated by the vascular wall composition, such as a reduced foam cell presence or fibrillated tissue in the vessel wall. In yet other embodiments, such improved vascular healing is demonstrated by improved inflammatory markers in the vascular wall. In other embodiments, such improved healing may be demonstrated by the retardation, halting or reversing of the fibrosis process in the liver or other organs or tissues.

The improved profile resulting from treatment with the compositions of the present invention may also be demonstrated by a differentiated impact on blood/serum/plasma lipid and lipoprotein levels in a mammal; these include, but are not limited to: Triglycerides (TG), total-cholesterol, non-HDL-cholesterol, LDL-cholesterol, VLDL-cholesterol, apolipoprotein B, apolipoprotein A, apolipoprotein C-III, HDL-cholesterol, and Lp-PLA2. The compositions of the present invention may also be used to provide a beneficial impact on the one or more of the following: apolipoprotein A-I (apo A-I), apolipoprotein B (apo B), apo A-I/apo B ratio, lipoprotein(a) (Lp[a]), lipoprotein-associated phospholipase A2 (Lp-PLA2), low density lipoprotein (LDL) particle number and size, oxidized LDL, C-reactive protein (CRP), high sensitivity C-reactive protein (HSCRP), intracellular adhesion molecule-1 (ICAM-1), E-selectin, P-selectin, vascular cell adhesion molecule 1 (VCAM-1) or cluster of differentiation 106 (CD106), interleukin-1β (IL-1β), interleukin-2 (IL-2), interleukin-6 (IL-6), interleukin-8 (IL-8), interleukin-10 (IL-10), interleukin-12 (IL-12), interleukin-15 (IL-15), interleukin-18 (IL-18), tumor necrosis factor-alpha (TNF-α), tumor necrosis factor-beta (TNF-β), plasminogen activator inhibitor-1 (PAI-1), homocysteine, thromboxane B2 (TXB2), thromboxane A2 (TXA2), 2,3-dinor thromboxane B2, free fatty acids (FFA), serum amyloid A1, serum amyloid A2, serum amyloid A3, serum amyloid A4, thiobarbituric acid (TBA) reacting material, adiponectin (GBP-28), hemoglobin A1c (HbA1c), macrophage colony stimulating factor (M-CSF), granulocyte macrophage colony stimulating factor (GM-CSF), fibrinogen, fibrin D-dimer, platelet derived-microparticles, mean platelet volume (MPV), platelet subpopulations, heart rate, systolic and diastolic blood pressure, nuclear factor kappa-light-chain enhancer of activated B cells (NF-κβ), adenosine diphosphate induced platelet aggregation, platelet endothelial cell adhesion molecule (PECAM-1), vitronectin receptor (αᵥβᵥ), and glycoprotein IIb/IIIa (gpIIIb/IIIa). The compositions of the present invention may also be used in methods of treating, preventing, and reducing symptoms associated with conditions associated with the above.

The compositions of the present invention may also be taken as a general nutritional supplement.

On a EPA+DPA daily dose basis, the compositions of the present invention are preferably provided in a dose of between 100 mg and 10,100 mg/day, alternatively between 200 mg and 8,100 mg/day, alternatively between 300 mg and 6,100 mg/day, alternatively between 400 mg and 5,100 mg/day, alternatively between 500 mg and 4,100 mg/day.

On a EPA+HPA+DPA daily dose basis, the compositions of the present invention are preferably provided in a dose of between 100 mg and 10,100 mg/day, alternatively between 200 mg and 8,100 mg/day, alternatively between 300 mg and 6,100 mg/day, alternatively between 400 mg and 5.100 mg/day, alternatively between 500 mg and 4,100 mg/day.

On a DPA+DHA daily dose basis, the compositions of the present invention are preferably provided in a dose of between 100 mg and 10,100 mg/day, alternatively between 200 mg and 8,100 mg/day, alternatively between 300 mg and 6,100 mg/day, alternatively between 400 mg and 5,100 mg/day, alternatively between 500 mg and 4,100 mg/day.

The formulation may be a single daily dose preparation to give in one dose the above intakes, or may be in convenient divided doses, for example, a daily dose formed of two to four soft gelatin or other dosage forms, each containing 300-1500 mg of EPA+DPA or EPA+DPA+HPA or DPA+DHA in any form embodied in the present invention.

Flavourants or emulsifiers may be included, for instance, to make the preparation palatable. Other conventional additives, diluents and excipients may be present. The preparation for ingestion may be in the form of a capsule, a dry powder, a tablet, a solution, an oil, an emulsion or any other appropriate form. The capsules may be hard or soft gelatin capsules, agar capsules, or any other appropriate capsule.

Use of the formulations of the invention in the manufacture of a medicament for the treatment or prevention of any disease or disorder, including those mentioned above, is included in the present invention.

The omega-3 fatty acid composition optionally includes chemical antioxidants, such as alpha tocopherol, which are administered in pure form or suspended in a vegetable oil, such as soybean oil or corn oil.

The blended fatty acid compositions may then be incorporated into any appropriate dosage form for oral, enteral, parenteral, rectal, vaginal, dermal or other route of administration. Soft or hard gelatin capsules, flavoured oil blends, emulsifiers or other liquid forms, and microencapsulate powders or other dry form vehicles are all appropriate ways of administering the products.

The formulated final drug product containing the omega-3 fatty acid composition may be administered to a mammal or patient in need thereof in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin. The combination product may also be contained in a liquid suitable for injection or infusion.

Example pharmaceutical grade finished dosage forms: (a) Soft or hard gelatin capsules each containing 500 mg or 1000 mg of a mix 20 parts of DHA as a free fatty acid to 1 parts of DPA as a free fatty acid; (b) As in (a) but where the DHA and DPA free fatty acids are replaced with the fatty acids in any other appropriate bioassimilable form such as the ethyl esters; (c) As in (a)-(b) but where the material is in the form of a microencapsulated powder which can be used as a powder or compressed into tablets. Such powders may be prepared by a variety of technologies known to those skilled in the art; (d) As in (a)-(b) but where the formulation is a liquid or emulsion, appropriately flavoured for palatable oral administration; (e) As in (a)-(b) but where the material is formulated into a pharmaceutically acceptable vehicle appropriate for topical application such as a cream or ointment.

The omega-3 compositions of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

The term “pharmaceutically acceptable vehicle,” as used herein, includes any of the following: a solution where the first API and optional other ingredients are wholly dissolved in a solubilizer (e.g., a pharmaceutically acceptable solvent or mixture of solvents), wherein the solution remains in clear liquid form at about room temperature; a suspension; an oil; or a semi-solid, wherein the first API and optionally other ingredients are dissolved wholly or partially in a solubilizer (an emulsion, cream, etc.).

A “pharmaceutical grade finished dosage form” as used herein may be construed as a unit dose form suitable for administration to, for example, human or animal subjects, and having content uniformity acceptable to regulatory authorities. For example, under the USP requirements for content uniformity, a pharmaceutical grade finished dosage form should have an amount of API within the range of 85% to 115% of the desired dosage and an RSD less than or equal to 6.0%. In addition, a pharmaceutical grade finished dosage form must be stable (i.e., have a “shelf life”) for a pharmaceutically acceptable duration of time, preferably at least six months, alternatively at least one year, or at least two years, when stored at room temperature (about 23 degree Celcius to 27 degree Celcius, preferably about 25 degree Celcius) and 60% relative humidity. Typically, stability is determined by physical appearance and/or chemical modification of the ingredients, in accordance with standards well-known in the pharmaceutical arts, including those documented in ICH guidelines.

The omega-3 fatty acid dosage form optionally includes chemical antioxidants, such as alpha tocopherol, oils, such as soybean oil and partially hydrogenated vegetable oil, and lubricants such as fractionated coconut oil, lecithin and a mixture of the same.

### EXAMPLES

### Example 1

A composition according to the present prevention is prepared by mixing and homogenizing in a ratio of 98:2 the intermediates MEGAPEX E90D00EE (90% EPA ethyl ester), and MAXOMEGA DPA95 FFA (≧95% DPA synthetic fatty acid produced from EPA ethyl ester concentrate) converted to ethyl ester, respectively. These intermediates were prepared and commercially offered for sale by Chemport Korea (MEGAPEX) and Equateq Ltd from Scotland, UK (MAXOMEGA). The relative amounts of fatty acids present in the starting intermediates and in the resulting novel composition are listed in Table 1 below. The resulting novel composition comprises 89.10% EPA, 1.95% DPA, 0.19% HPA, 91.24% omega-3-pentaenoic acids, less than 0.01% DHA, 91.24% omega-3-pentaenoic acids, 93.09% total omega-3 fatty acids, 3.15% ARA and 3.57% omega-6 fatty acids (all Area %).

TABLE 1
Fatty acid Composition (Area %) of intermediates
and novel composition according to Example 1

| | 98.0% | 2.0% | |
|---|---|---|---|
| | Megapex | Maxomega | Novel |
| Fatty Acid | E90D00EE | DPA95FFA => EE | Composition |
| c18:0 | 0.05 | 0 | 0.05 |
| c18:1n9 | 0.06 | 0 | 0.06 |
| c18:1n7 | 0.02 | 0 | 0.02 |
| c18:2n6 | 0.01 | 0 | 0.01 |
| c18:3n6 | 0.02 | 0 | 0.02 |
| c18:3n3 | 0.03 | 0 | 0.03 |
| c18:4n3 | 0.42 | 0 | 0.41 |
| c18:4n1 | 0.07 | 0 | 0.07 |
| c20:0 | 0 | 0 | 0.00 |
| c20:1n11 | 0 | 0 | 0.00 |
| c20:1n9 | 0 | 0 | 0.00 |
| c20:1n7 | 0 | 0 | 0.00 |
| c20:2n6 | 0.25 | 0 | 0.25 |
| c20:3n9 | 0 | 0 | 0.00 |
| c20:3n6 | 0.15 | 0 | 0.15 |
| c21:0 | 0 | 0 | 0.00 |
| c20:4n6 | 3.21 | 0 | 3.15 |
| c20:3n3 | 0 | 0 | 0.00 |
| c20:4n3 | 1.44 | 0 | 1.41 |
| c20:5n3 | 90.92 | 0 | 89.10 |
| c22:0 | 0.3 | 0 | 0.29 |
| c22:1n11 | 0.07 | 0 | 0.07 |
| c22:1n9 | 0.18 | 0 | 0.18 |
| c22:1n7 | 0.19 | 0 | 0.19 |
| c21.5n3 | 0.19 | 0 | 0.19 |
| c22:5n6 | 0 | 0 | 0.00 |
| c22:5n3 | 0 | 97.27 | 1.95 |
| c22:6n3 | 0 | 0 | 0.00 |
| c24:0 | 0 | 0.33 | 0.01 |
| OTHER | 2.42 | 2.4 | 2.42 |
| | 100 | 100 | 100 |

### Example 2

A composition according to the present prevention is prepared by mixing and homogenizing in a ratio of 96:4 the intermediates MEGAPEX E90D00EE (90% EPA ethyl ester), and MAXOMEGA DPA95 FFA (≧95% DPA synthetic fatty acid produced from EPA ethyl ester concentrate), converted to ethyl ester, respectively. These intermediates were prepared and commercially offered for sale by Chemport Korea (MEGAPEX) and Equateq Ltd from Scotland, UK (MAXOMEGA). The relative amounts of fatty acids present in the starting intermediates and in the resulting novel composition is listed in Table 2 below. The resulting novel composition comprises 87.28% EPA, 3.89% DPA, 0.18% HPA, 91.35% omega-3-pentaenoic acids, less than 0.01% DHA, 93.17% total omega-3 fatty acids and 3.49% omega-6 fatty acids (all Area %).

TABLE 2
Fatty acid Composition (Area %) of intermediates
and novel composition according to Example 2

| | 96.0% | 4.0% | |
|---|---|---|---|
| | Megapex | Maxomega | Novel |
| Fatty Acid | E90D00EE | DPA95FFA => EE | Composition |
| c18:0 | 0.05 | 0 | 0.05 |
| c18:1n9 | 0.06 | 0 | 0.06 |
| c18:1n7 | 0.02 | 0 | 0.02 |
| c18:2n6 | 0.01 | 0 | 0.01 |
| c18:3n6 | 0.02 | 0 | 0.02 |
| c18:3n3 | 0.03 | 0 | 0.03 |
| c18:4n3 | 0.42 | 0 | 0.40 |
| c18:4n1 | 0.07 | 0 | 0.07 |
| c20:0 | 0 | 0 | 0.00 |
| c20:1n11 | 0 | 0 | 0.00 |
| c20:1n9 | 0 | 0 | 0.00 |
| c20:1n7 | 0 | 0 | 0.00 |
| c20:2n6 | 0.25 | 0 | 0.24 |
| c20:3n9 | 0 | 0 | 0.00 |
| c20:3n6 | 0.15 | 0 | 0.14 |
| c21:0 | 0 | 0 | 0.00 |
| c20:4n6 | 3.21 | 0 | 3.08 |
| c20:3n3 | 0 | 0 | 0.00 |
| c20:4n3 | 1.44 | 0 | 1.38 |
| c20:5n3 | 90.92 | 0 | 87.28 |
| c22:0 | 0.3 | 0 | 0.29 |
| c22:1n11 | 0.07 | 0 | 0.07 |
| c22:1n9 | 0.18 | 0 | 0.17 |
| c22:1n7 | 0.19 | 0 | 0.18 |
| c21:5n3 | 0.19 | 0 | 0.18 |
| c22:5n6 | 0 | 0 | 0.00 |
| c22:5n3 | 0 | 97.27 | 3.89 |
| c22:6n3 | 0 | 0 | 0.00 |
| c24:0 | 0 | 0.33 | 0.01 |
| OTHER | 2.42 | 2.4 | 2.42 |
| | 100 | 100 | 100 |

### Example 3

A composition according to the present prevention is prepared by mixing and homogenizing in a ratio of 94:6 the intermediates MEGAPEX E90D00EE (90% EPA ethyl ester), and MAXOMEGA DPA95 FFA (≧95% DPA synthetic fatty acid produced from EPA ethyl ester concentrate) converted to ethyl ester, respectively. These intermediates were prepared and commercially offered for sale by Chemport Korea (MEGAPEX) and Equateq Ltd from Scotland, UK (MAXOMEGA). The relative amounts of fatty acids present in the starting intermediates and in the resulting novel composition are listed in table 3 below. The resulting novel composition comprises 85.46% EPA, 5.84% DPA, 0.18% HPA, 91.48% omega-3-pentaenoic acids, less than 0.01% DHA, 93.26% total omega-3 fatty acids, 3.02% ARA, and 3.42% omega-6 fatty acids (all Area %).

TABLE 3
Fatty acid Composition (Area %) of intermediates
and novel composition according to Example 3

| | 94.0% | 6.0% | |
|---|---|---|---|
| | Megapex | Maxomega | Novel |
| Fatty Acid | E90D00EE | DPA95FF4 => EE | Composition |
| c18:0 | 0.05 | 0 | 0.05 |
| c18:1n9 | 0.06 | 0 | 0.06 |
| c18:1n7 | 0.02 | 0 | 0.02 |
| c18:2n6 | 0.01 | 0 | 0.01 |
| c18:3n6 | 0.02 | 0 | 0.02 |
| c18:3n3 | 0.03 | 0 | 0.03 |
| c18:4n3 | 0.42 | 0 | 0.39 |
| c18:4n1 | 0.07 | 0 | 0.07 |
| c20:0 | 0 | 0 | 0.00 |
| c20:1n11 | 0 | 0 | 0.00 |
| c20:1n9 | 0 | 0 | 0.00 |
| c20:1n7 | 0 | 0 | 0.00 |
| c20:2n6 | 0.25 | 0 | 0.24 |
| c20:3n9 | 0 | 0 | 0.00 |
| c20:3n6 | 0.15 | 0 | 0.14 |
| c21:0 | 0 | 0 | 0.00 |
| c20:4n6 | 3.21 | 0 | 3.02 |
| c20:3n3 | 0 | 0 | 0.00 |
| c20:4n3 | 1.44 | 0 | 1.35 |
| c20:5n3 | 90.92 | 0 | 85.46 |
| c22:0 | 0.3 | 0 | 0.28 |
| c22:1n11 | 0.07 | 0 | 0.07 |
| c22:1n9 | 0.18 | 0 | 0.17 |
| c22:1n7 | 0.19 | 0 | 0.18 |
| c21:5n3 | 0.19 | 0 | 0.18 |
| c22:5n6 | 0 | 0 | 0.00 |
| c22:5n3 | 0 | 97.27 | 5.84 |
| c22:6n3 | 0 | 0 | 0.00 |
| c24:0 | 0 | 0.33 | 0.02 |
| OTHER | 2.42 | 2.4 | 2.42 |
| | 100 | 100 | 100 |

### Example 4

A composition according to the present prevention is prepared by mixing and homogenizing in a ratio of 75:25 the intermediates MEGAPEX E90D00EE (90% EPA ethyl ester), and MAXOMEGA DPA95 FFA (≧95% DPA synthetic fatty acid produced from EPA ethyl ester concentrate, converted to ethyl ester, respectively. These intermediates were prepared and commercially offered for sale by Chemport Korea (MEGAPEX) and Equateq Ltd from Scotland, UK (MAXOMEGA). The relative amounts of fatty acids present in the starting intermediates and in the resulting novel composition is listed in table 4 below. The resulting novel composition comprises 68.10% EPA, 24.32% DPA, 0.19% HPA, 92.65% omega-3-pentaenoic acids, less than 0.01% DHA, 94.07% total omega-3 fatty acids, 2.41% ARA and 2.73% omega-6 fatty acids (all Area %).

TABLE 4
Fatty acid Composition (Area %) of intermediates
and novel composition according to Example 4

| | 75.0% | 25.0% | |
|---|---|---|---|
| | Megapex | Maxomega | Novel |
| Fatty Acid | E90D00EE | DPA95FFA => EE | Composition |
| c18:0 | 0.05 | 0 | 0.04 |
| c18:1n9 | 0.06 | 0 | 0.05 |
| c18:1n7 | 0.02 | 0 | 0.02 |
| c18:2n6 | 0.01 | 0 | 0.01 |
| c18:3n6 | 0.02 | 0 | 0.02 |
| c18:3n3 | 0.03 | 0 | 0.02 |
| c18:4n3 | 0.42 | 0 | 0.32 |
| c18:4n1 | 0.07 | 0 | 0.05 |
| c20:0 | 0 | 0 | 0.00 |
| c20:1n11 | 0 | 0 | 0.00 |
| c20:1n9 | 0 | 0 | 0.00 |
| c20:1n7 | 0 | 0 | 0.00 |
| c20:2n6 | 0.25 | 0 | 0.19 |
| c20:3n9 | 0 | 0 | 0.00 |
| c20:3n6 | 0.15 | 0 | 0.11 |
| c21:0 | 0 | 0 | 0.00 |
| c20:4n6 | 3.21 | 0 | 2.41 |
| c20:3n3 | 0 | 0 | 0.00 |
| c20:4n3 | 1.44 | 0 | 1.08 |
| c20:5n3 | 90.92 | 0 | 68.19 |
| c22:0 | 0.3 | 0 | 0.23 |
| c22:1n11 | 0.07 | 0 | 0.05 |
| c22:1n9 | 0.18 | 0 | 0.14 |
| c22:1n7 | 0.19 | 0 | 0.14 |
| c21:5n3 | 0.19 | 0 | 0.14 |
| c22:5n6 | 0 | 0 | 0.00 |
| c22:5n3 | 0 | 97.27 | 24.32 |
| c22:6n3 | 0 | 0 | 0.00 |
| c24:0 | 0 | 0.33 | 0.08 |
| OTHER | 2.42 | 2.4 | 2.42 |
| | 100 | 100 | 100 |

### Example 5

A composition according to the present prevention is prepared by mixing and homogenizing in a ratio of 60:40 the intermediates KD-PharmaKD-PUR 900EE and MAXOMEGA DPA95 FFA converted to ethyl ester, respectively. These intermediates were prepared and commercially offered for sale by KD-Pharma Germany (KD-Pharma) and Equateq Ltd from Scotland, UK (MAXOMEGA). The relative amounts of fatty acids present in the starting intermediates and in the resulting novel composition is listed in table 5 below. The resulting novel composition comprises 55.74% EPA, 39.26% DPA, 2.39% HPA, 97.44% omega-3-pentaenoic acids, and 98.06% total omega-3 fatty acids (all Area %).

TABLE 5
Fatty acid Composition (Area %) of intermediates
and novel composition according to Example 5

| | | 40.0% | |
|---|---|---|---|
| | 60.0% | Maxomega | Novel |
| Fatty Acid | KD-Pur 900EE | DPA95FFA => EE | Composition |
| c18:0 | 0 | 0 | 0.00 |
| c18:1n9 | 0 | 0 | 0.00 |
| c18:1n7 | 0 | 0 | 0.00 |
| c18:2n6 | 0 | 0 | 0.00 |
| c18:3n6 | 0 | 0 | 0.00 |
| c18:3n3 | 0 | 0 | 0.00 |
| c18:4n3 | 0 | 0 | 0.00 |
| c18:4n1 | 0 | 0 | 0.00 |
| c20:0 | 0 | 0 | 0.00 |
| c20:1n11 | 0 | 0 | 0.00 |
| c20:1n9 | 0 | 0 | 0.00 |
| c20:1n7 | 0 | 0 | 0.00 |
| c20:2n6 | 0 | 0 | 0.00 |
| c20:3n9 | 0 | 0 | 0.00 |
| c20:3n6 | 0 | 0 | 0.00 |
| c21:0 | 0 | 0 | 0.00 |
| c20:4n6 | 0 | 0 | 0.00 |
| c20:3n3 | 0 | 0 | 0.00 |
| c20:4n3 | 1.04 | 0 | 0.62 |
| c20:5n3 | 92.99 | 0 | 55.79 |
| c22:0 | 0 | 0 | 0.00 |
| c22:1n11 | 0 | 0 | 0.00 |
| c22:1n9 | 0 | 0 | 0.00 |
| c22:1n7 | 0 | 0 | 0.00 |
| c21:5n3 | 3.98 | 0 | 2.39 |
| c22:5n6 | 0 | 0 | 0.00 |
| c22:5n3 | 0.58 | 97.27 | 39.26 |
| c22:6n3 | 0 | 0 | 0.00 |
| c24:0 | 0 | 0.33 | 0.13 |
| OTHER | 1.41 | 2.4 | 1.81 |
| | 100.00 | 100 | 100.00 |

### Example 6

A composition according to the present prevention is prepared by mixing and homogenizing in a ratio of 96:4 the intermediates KD-PUR 900EE KD-Pharma and MAXOMEGA DPA95 FFA converted to ethyl ester, respectively. These intermediates were prepared and commercially offered for sale by KD-Pharma Germany (KD-Pharma) and Equateq Ltd from Scotland, UK (MAXOMEGA). The relative amounts of fatty acids present in the starting intermediates and in the resulting novel composition is listed in table 6 below. The resulting novel composition comprises 89.27% EPA, 4.45% DPA, 3.82% HPA, 97.54% omega-3-pentaenoic acids, and 98.54% total omega-3 fatty acids (all Area %).

TABLE 6
Fatty acid Composition (Area %) of intermediates
and novel composition according to Example 6

| | | 4.0% | |
|---|---|---|---|
| | 96.0% | Maxomega | Novel |
| Fatty Acid | KD-Pur 900EE | DPA95FFA => EE | Composition |
| c18:0 | 0 | 0 | 0.00 |
| c18:1n9 | 0 | 0 | 0.00 |
| c18:1n7 | 0 | 0 | 0.00 |
| c18:2n6 | 0 | 0 | 0.00 |
| c18:3n6 | 0 | 0 | 0.00 |
| c18:3n3 | 0 | 0 | 0.00 |
| c18:4n3 | 0 | 0 | 0.00 |
| c18:4n1 | 0 | 0 | 0.00 |
| c20:0 | 0 | 0 | 0.00 |
| c20:1n11 | 0 | 0 | 0.00 |
| c20:1n9 | 0 | 0 | 0.00 |
| c20:1n7 | 0 | 0 | 0.00 |
| c20:2n6 | 0 | 0 | 0.00 |
| c20:3n9 | 0 | 0 | 0.00 |
| c20:3n6 | 0 | 0 | 0.00 |
| c21:0 | 0 | 0 | 0.00 |
| c20:4n6 | 0 | 0 | 0.00 |
| c20:3n3 | 0 | 0 | 0.00 |
| c20:4n3 | 1.04 | 0 | 1.00 |
| c20:5n3 | 92.99 | 0 | 89.27 |
| c22:0 | 0 | 0 | 0.00 |
| c22:1n11 | 0 | 0 | 0.00 |
| c22:1n9 | 0 | 0 | 0.00 |
| c22:1n7 | 0 | 0 | 0.00 |
| c21:5n3 | 3.98 | 0 | 3.82 |
| c22:5n6 | 0 | 0 | 0.00 |
| c22:5n3 | 0.58 | 97.27 | 4.45 |
| c22:6n3 | 0 | 0 | 0.00 |
| c24:0 | 0 | 0.33 | 0.01 |
| OTHER | 1.41 | 2.4 | 1.45 |
| | 100.00 | 100 | 100.00 |

### Example 7

The ethyl ester composition of Example 4 may be converted into a free fatty acid composition with essentially the same fatty acid composition according to “Conversion Method EE to FFA” below. This method is indiscriminate with respect to the type, degree of saturation or length of fatty acid if performed for an adequate amount of time under the described conditions.

Conversion Method EE to FFA
1. Fatty Acid Ethyl Ester (FAEE GMP, approx. 3 mmol/g) oil is brought into a closed heated/cooled reaction chamber under nitrogen atmosphere (preferably with pressure control), and heated to 50-60 degree Celcius under stirring.
2. 2M NaOH solution in water is added under firm stirring to ensure phase mixing (est. 2-3×FAEE w/w) and stir until no ethyl ester is presence (est. 2-4 hrs). Test ethyl ester presence at lab scale/in process with TLC (hexanes/EtOAc 9:1) and with EP GC method to confirm reaction completion under GMP.
3. Under cooling (keep mixture below 70 degree Celcius), add 6M HCl in water (est. <1 hr) until slightly acid (˜pH3-4). It may be necessary to control pressure to prevent excessive foaming. Then halt stirring, give time to let phases separate, and remove water phase from bottom (keep oil protected from oxygen, apply nitrogen atmosphere blanket).
4. Add demineralized water (est. 2-3×FAEE w/w) and wash out NaCl and ethanol from oil under firm stirring (est. ˜1 hr). Halt stirring, give time to let phases separate, and remove water phase from bottom (keep oil protected from oxygen, apply nitrogen atmosphere blanket).
5. Repeat Step 4 several times (˜2×) to remove ethanol and NaCl.
6. Remove water and remaining ethanol [determine in-process controls], confirm under GMP with USP residual solvent method (target: ethanol <100 ppm) by stirring oil while applying vacuum 10-50 mbar (with solvent trap) and heat oil (70-80 degree celcius) until water/ethanol target is met (est. 2-4 hrs).
7. Add anti-oxidants (i.e. alpha-D-tocopherol, USP, target 4 mg/g) and/or other excipients.
8. All reagents and excipients USP grade.

### Example 8

The ethyl ester composition of Example 3 is converted into a free fatty acid composition with essentially the same fatty acid composition according to “Conversion Method EE to FFA” above. This method is indiscriminate with respect to the type, degree of saturation or length of fatty acid if performed for an adequate amount of time under the described conditions.

### Example 9

The ethyl ester composition of Example 6 is converted into a free fatty acid composition with essentially the same fatty acid composition according to “Conversion Method EE to FFA” above. This method is indiscriminate with respect to the type, degree of saturation or length of fatty acid if performed for an adequate amount of time under the described conditions.

### Example 10

The composition of Example 4 is formulated into a soft gelatin capsule. Prior to encapsulation, an anti-oxidant preparation (composed of 4000 mg alpha-D-tocopherol in one liter of corn oil; corn oil is a triglyceride low in omega-3) is added to the composition of Example 4, by mixing and homogenizing 100 mL of this anti-oxidant preparation into 100 liters of the oil composition of Example 4 followed by thorough homogenization. The resulting pre-encapsulation formulated oil contains approximately 4 mg/gram alpha-D-tocopherol. Subsequently, the formulated oil is encapsulated into soft gelatin capsules with printed logo according to general methods typically used by Accucaps in Canada for fish oils or by any other documented and operational encapsulation method. The fill mass of the oil is approximately 1.08 gram/capsule, providing a dose of approximately 1000 mg omega-3-pentaenoic-acids ethyl esters per capsule. Finally, the capsules are bottled in HDPE bottles with induction seal and child resistant cap.

### Example 11

The composition of Example 8 is formulated into a soft gelatin capsule. Prior to encapsulation, an anti-oxidant preparation (composed of 4000 mg alpha-D-tocopherol in one liter of corn oil; corn oil is a triglyceride low in omega-3) is added to the composition of Example 4, by mixing and homogenizing 100 mL of this anti-oxidant preparation into 100 liters of the oil composition of Example 4 followed by thorough homogenization. The resulting pre-encapsulation formulated oil contains approximately 4 mg/gram alpha-D-tocopherol. Subsequently, the formulated oil is encapsulated into soft gelatin capsules with printed logo according to general methods typically used by Banner in High Point, N.C., for fish oils or by any other documented and operational encapsulation method. The fill mass of the oil is approximately 1.09 gram/capsule, providing a dose of approximately 1000 mg omega-3-pentaenoic-acids per capsule. Finally, the capsules are bottled in HOPE bottles with induction seal and child resistant cap.

### Example 12

The composition of Example 5 is formulated into a soft gelatin capsule. Prior to encapsulation, an anti-oxidant preparation (composed of 4000 mg alpha-D-tocopherol in one liter of corn oil; corn oil is a triglyceride low in omega-3) is added to the composition of Example 4, by mixing and homogenizing 100 mL of this anti-oxidant preparation into 100 liters of the oil composition of Example 4 followed by thorough homogenization. The resulting pre-encapsulation formulated oil contains approximately 4 mg/gram alpha-D-tocopherol. Subsequently, the formulated oil is encapsulated into soft gelatin capsules with printed logo according to general methods typically used by Catalent in St. Petersburg, Fla., for fish oils or by any other documented and operational encapsulation method. The fill mass of the oil is approximately 1.05 gram/capsule, providing a dose of approximately 1000 mg omega-3-pentaenoic-acids ethyl esters per capsule. Finally, the capsules are bottled in HDPE bottles with induction seal and child resistant cap.

### Example 13

The composition of Example 9 is formulated into a soft gelatin capsule. Prior to encapsulation, an anti-oxidant preparation (composed of 4000 mg alpha-D-tocopherol in one liter of corn oil; corn oil is a triglyceride low in omega-3) is added to the composition of Example 4, by mixing and homogenizing 100 mL of this anti-oxidant preparation into 100 liters of the oil composition of Example 4 followed by thorough homogenization. The resulting pre-encapsulation formulated oil contains approximately 4 mg/gram alpha-D-tocopherol. Subsequently, the formulated oil is encapsulated into soft gelatin capsules with printed logo according to general methods typically used by Banner in High Point, N.C., for fish oils or by any other documented and operational encapsulation method. The fill mass of the oil is 1.06 gram/capsule, providing a dose of approximately 1000 mg omega-3-pentaenoic-acids per capsule. Finally, the capsules are bottled in HOPE bottles with induction seal and child resistant cap.

### Example 14

The following is an example formulation.

COMPOSITION 1

| | Minimum | Maximum | Target |
|---|---|---|---|
| Composition | (mg/g) | (mg/g) | (mg/g) |
| Omega-3 pentaenoic acid | 870 | 990 | 920 |
| Eicosapentaenoic acid (EPA) | 750 | 950 | 830 |
| Heneicosapentaenoic acid (HPA) | 5 | 70 | 40 |
| Docosapentaenoic acid (DPA) | 50 | 130 | 90 |
| Docosahexaenoic acid (DHA) | | 40 | 20 |

In COMPOSITION 1, the EPA:DPA ratio is between 13 and 190, the EPA:DPA ratio is between 8 and 15, the HPA:DPA ration between 0.05 and 1, the DPA:DHA ratio more than 2.4, preferably more than 4, more preferably more than 6, most preferably more than 10, and the EPA:DHA ratio more than 32, preferably more than 38, more preferably more than 80, most preferably more than 95. The EPA, HPA, DPA and DHA may be composed as a glyceride (such as triglyceride), an ester (such as ethyl ester), or a free fatty acid.

### Example 15

The following is an example formulation

COMPOSITION 2

| | Minimum | Maximum | Target |
|---|---|---|---|
| Composition | (mg/g) | (mg/g) | (mg/g) |
| Omega-3 pentaenoic acid | 900 | 980 | 940 |
| Eicosapentaenoic acid (EPA) | 15 | 60 | 30 |
| Heneicosapentaenoic acid (HPA) | 5 | 60 | 30 |
| Docosapentaenoic acid (DPA) | 800 | 950 | 880 |
| Docosahexaenoic acid (DHA) | | 25 | <10 |

In COMPOSITION 2, the EPA:DPA ratio is between 0.25 and 12, the DPA:HPA ratio is between 13 and 63, the DPA:HPA ration between 13 and 190, the DPA:DHA ratio more than 32, preferably more than 38, more preferably more than 80, most preferably more than 95, and the EPA:DHA ratio more than 0.6, preferably more than 1.5, more preferably more than 2.4, most preferably more than 6. The EPA, HPA, DPA and DHA may be composed as a glyceride (such as triglyceride), an ester (such as ethyl ester), or a free fatty acid.

### Example 16

The following is an example formulation.

COMPOSITION 3

| | Minimum | Maximum | Target |
|---|---|---|---|
| Composition | (mg/g) | (mg/g) | (mg/g) |
| Docosapentaenoic acid (DPA n-3) | 800 | 990 | 920 |

The DPA may be composed as a glyceride (such as triglyceride), an ester (such as ethyl ester), or a free fatty acid.

### Example 17

The following is an example formulation.

COMPOSITION 4

| | Minimum | Maximum | Target |
|---|---|---|---|
| Composition | (mg/g) | (mg/g) | (mg/g) |
| Docosapentaenoic acid (DPA n-3) | 650 | 950 | 800 |
| Docosahexaenoic acid (DHA n-3) | 50 | 350 | 200 |

### Example 18

The following is an example formulation.

COMPOSITION 5

| | Minimum | Maximum | Target |
|---|---|---|---|
| Composition | (mg/g) | (mg/g) | (mg/g) |
| Docosapentaenoic acid (DPA n-3) | 350 | 650 | 500 |
| Docosahexaenoic acid (DHA n-3) | 350 | 650 | 500 |

### Example 19

The following is an example formulation.

COMPOSITION 6

| | Minimum | Maximum | Target |
|---|---|---|---|
| Composition | (mg/g) | (mg/g) | (mg/g) |
| Docosapentaenoic acid (DPA n-3) | 50 | 350 | 200 |
| Docosahexaenoic acid (DHA n-3) | 650 | 950 | 800 |

### Example 20

A patient is diagnosed with NAFLD. Thereupon, the patient may be initiated on daily treatment with one of the encapsulated compositions according to Examples 10, 11, 12 or 13, or Compostions 1, 2, 3, 4, 5, or 6. The treatment results in significant clinical improvement, including reduction in liver enzyme levels.

### Example 21

The following describes a study to determine the effects of composition of the present invention.

The study tests the effects of preparations comprising about 90% DPA and DHA on Zucker rats. The rats are placed in 5 active treatment groups: (1) a high DPA/low DHA preparation (ratio of DPA:DHA of about 4:1), at 200 mg/kg, (2) a low DPA/high DHA preparation (ratio of DPA:DHA of about 1:4) at 200 mg/kg, (3) a DPA/DHA preparation at 50 mg/kg, (4) a DPA/DHA preparation at 200 mg/kg, and (5) a DPA/DHA preparation at 600 mg/kg. The effect with these treatment groups is compared to inactive control (water) and active controls bile acid and pioglotazone. The treatment duration is 8 to 12 weeks, with final bleed and full necropsy upon sacrificing the animals at study termination. Liver, and select other tissues are frozen. Part of the liver is prepared for histology. Effect on gene expression by mRNA probe is used to test the effects of the treatments on the regulation of fatty liver disease associated genes. The study shows a beneficial effect.

### DESCRIPTION OF THE EMBODIMENTS

1. A fatty acid composition comprising at least 50% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
2. A fatty acid composition comprising at least 60% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
3. A fatty acid composition comprising at least 70% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
4. A fatty acid composition comprising at least 75% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
5. A fatty acid composition comprising at least 80% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
6. A fatty acid composition comprising at least 85% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
7. A fatty acid composition comprising at least 90% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20:1.
8. A fatty acid composition comprising at least 95% omega-3-fatty acids, salts or derivatives thereof, while comprising eicosapentaenoic acid (EPA; C20:5-n3) and docosapentaenoic acid (DPA; C22:5-n3) and wherein the EPA:DHA ratio is higher than 20.1.
9. A composition according to one of the preferred embodiments 1 through 8, comprising at least 2% docosapentaenoic acid (DPA; C22:5-n3).
10. A composition according to one of the preferred embodiments 1 through 8, comprising at least 4% docosapentaenoic acid (DPA; C22:5-n3).
11. A composition according to one of the preferred embodiments 1 through 8, comprising at least 5% docosapentaenoic acid (DPA; C22:5-n3).
12. A composition according to one of the preferred embodiments 1 through 8, comprising at least 6% docosapentaenoic acid (DPA; C22:5-n3).
13. A composition according to one of the preferred embodiments 1 through 8, comprising at least 7% docosapentaenoic acid (DPA; C22:5-n3).
14. A composition according to one of the preferred embodiments 1 through 8, comprising at least 8% docosapentaenoic acid (DPA; C22:5-n3).
15. A composition according to one of the preferred embodiments 1 through 8, comprising at least 10% docosapentaenoic acid (DPA; C22:5-n3).
16. A composition according to one of the preferred embodiments 1 through 8, comprising at least 12% docosapentaenoic acid (DPA; C22:5-n3).
17. A composition according to one of the preferred embodiments 1 through 8, comprising at least 15% docosapentaenoic acid (DPA; C22:5-n3).
18. A composition according to one of the preferred embodiments 1 through 17, comprising no more than 95% EPA.
19. A composition according to one of the preferred embodiments 1 through 17, comprising no more than 10% omega-6 fatty acids.
20. A composition according to one of the preferred embodiments 1 through 17, comprising no more than 7% omega-6 fatty acids.
21. A composition according to one of the preferred embodiments 1 through 17, comprising no more than 5% omega-6 fatty acids.
22. A composition according to one of the preferred embodiments 1 through 17, comprising no more than 3% omega-6 fatty acids.
23. A composition according to one of the preferred embodiments 1 through 22, comprising no more than 5% arachidonic acid (C22:4-n6).
24. A composition according to one of the preferred embodiments 1 through 22, comprising no more than 4% arachidonic acid (C22:4-n6).
25. A composition according to one of the preferred embodiments 1 through 22, comprising no more than 3% arachidonic acid (C22:4-n6).
26. A composition according to one of the preferred embodiments 1 through 22, comprising no more than 2% arachidonic acid (C22:4-n6).
27. A composition according to one of the preferred embodiments 1 through 22, comprising no more than 1% arachidonic acid (C22:4-n6).
28. A composition according to one of the preferred embodiments 1 through 27, also comprising heneicosapentaenoic acid (C21:5-n3).
29. A composition according to one of the preferred embodiments 1 through 27, comprising at least 0.01% heneicosapentaenoic acid (C21:5-n3).
30. A composition according to one of the preferred embodiments 1 through 27, comprising at least 0.1% heneicosapentaenoic acid (C21:5-n3).
31. A composition according to one of the preferred embodiments 1 through 27, comprising at least 0.3% heneicosapentaenoic acid (C21:5-n3).
32. A composition according to one of the preferred embodiments 1 through 27, comprising at least 0.5% heneicosapentaenoic acid (C21:5-n3).
33. A composition according to one of the preferred embodiments 1 through 27, comprising at least 1% heneicosapentaenoic acid (C21:5-n3).
34. A composition according to one of the preferred embodiments 1 through 27, comprising at least 2% heneicosapentaenoic acid (C21:5-n3).
35. A composition according to one of the preferred embodiments 1 through 27, comprising at least 3% heneicosapentaenoic acid (C21:5-n3).
36. A composition according to one of the preferred embodiments 1 through 27, comprising at least 4% heneicosapentaenoic acid (C21:5-n3).
37. A composition according to one of the preferred embodiments 1 through 27, comprising at least 5% heneicosapentaenoic acid (C21:5-n3).
38. A composition according to one of the preferred embodiments 1 through 37, comprising no more than 5% omega-3 fatty acids that are not omega-3-pentaenoic acids.
39. A composition according to one of the preferred embodiments 1 through 37, comprising no more than 4% omega-3 fatty acids that are not omega-3-pentaenoic acids.
40. A composition according to one of the preferred embodiments 1 through 37, comprising no more than 3% omega-3 fatty acids that are not omega-3-pentaenoic acids.
41. A composition according to one of the preferred embodiments 1 through 37, comprising no more than 2% omega-3 fatty acids that are not omega-3-pentaenoic acids.
42. A composition according to one of the preferred embodiments 1 through 37, comprising no more than 1.5% omega-3 fatty acids that are not omega-3-pentaenoic acids.
43. A composition according to one of the preferred embodiments 1 through 37, comprising no more than 1.25% omega-3 fatty acids that are not omega-3-pentaenoic acids.
44. A composition according to one of the preferred embodiments 1 through 37, comprising no more than 1% omega-3 fatty acids that are not omega-3-pentaenoic acids.
45. A composition according to one of the preferred embodiments 1 through 44, wherein the EPA:DPA ratio is between 99:1 and 1:99.
46. A composition according to one of the preferred embodiments 1 through 44, wherein the EPA:DPA ratio is between 60:1 and 1:60.
47. A composition according to one of the preferred embodiments 1 through 44, wherein the EPA:DPA ratio is between 50:1 and 1:10.
48. A composition according to one of the preferred embodiments 1 through 44, wherein the EPA:DPA ratio is between 40:1 and 1:3.
49. A composition according to one of the preferred embodiments 1 through 44, wherein the EPA:DPA ratio is between 40:1 and 1:2.
50. A composition according to one of the preferred embodiments 1 through 44, wherein the EPA:DPA ratio is between 40:1 and 1:1.
51. A composition according to one of the preferred embodiments 1 through 44, wherein the EPA:DPA ratio is between 30:1 and 1:1.
52. A composition according to one of the preferred embodiments 1 through 44, wherein the EPA:DPA ratio is between 20:1 and 1:1.
53. A composition according to one of the preferred embodiments 1 through 44, wherein the EPA:DPA ratio is between 10:1 and 1:1.
54. A composition according to one of the preferred embodiments 1 through 44, wherein the EPA:DPA ratio is between 5:1 and 1:1.
55. A composition according to one of the preferred embodiments 1 through 44, wherein the EPA:DPA ratio is between 10:1 and 2:1.
56. A composition according to one of the preferred embodiments 1 through 44, wherein the EPA:DPA ratio is between 20:1 and 2:1.
57. A composition according to one of the preferred embodiments 1 through 44, wherein the EPA:DPA ratio is between 30:1 and 2:1.
58. A composition according to one of the preferred embodiments 1 through 44, wherein the EPA:DPA ratio is between 40:1 and 2:1.
59. A composition according to one of the preferred embodiments 1 through 44, wherein the EPA:DPA ratio is between 50:1 and 2:1.
60. A composition according to one of the preferred embodiments 1 through 44, wherein the EPA:DPA ratio is between 10:1 and 3:1.
61. A composition according to one of the preferred embodiments 1 through 44, wherein the EPA:DPA ratio is between 20:1 and 3:1.
62. A composition according to one of the preferred embodiments 1 through 44, wherein the EPA:DPA ratio is between 30:1 and 3:1.
63. A composition according to one of the preferred embodiments 1 through 44, wherein the EPA:DPA ratio is between 40:1 and 3:1.
64. A composition according to one of the preferred embodiments 1 through 44, wherein the EPA:DPA ratio is between 50:1 and 3:1.
65. A composition according to one of the preferred embodiments 1 through 44, wherein the EPA:DPA ratio is between 60:1 and 3:1.
66. A composition according to one of the preferred embodiments 1 through 44, wherein the EPA:DPA ratio is between 10:1 and 5.1.
67. A composition according to one of the preferred embodiments 1 through 44, wherein the EPA:DPA ratio is between 20:1 and 5:1.
68. A composition according to one of the preferred embodiments 1 through 44, wherein the EPA:DPA ratio is between 30:1 and 5:1.
69. A composition according to one of the preferred embodiments 1 through 44, wherein the EPA:DPA ratio is between 40:1 and 5:1.
70. A composition according to one of the preferred embodiments 1 through 44, wherein the EPA:DPA ratio is between 50:1 and 5:1.
71. A composition according to one of the preferred embodiments 1 through 44, wherein the EPA:DPA ratio is between 60:1 and 5:1.
72. A composition according to one of the preferred embodiments 1 through 44, wherein the EPA:DPA ratio is between 20:1 and 10:1.
73. A composition according to one of the preferred embodiments 1 through 44, wherein the EPA:DPA ratio is between 30:1 and 10:1.
74. A composition according to one of the preferred embodiments 1 through 44, wherein the EPA:DPA ratio is between 40:1 and 10:1.
75. A composition according to one of the preferred embodiments 1 through 44, wherein the EPA:DPA ratio is between 50:1 and 10:1.
76. A composition according to one of the preferred embodiments 1 through 44, wherein the EPA:DPA ratio is between 60:1 and 10:1.
77. A composition according to one of the preferred embodiments 1 through 44, wherein the EPA:DPA ratio is between 100:1 and 10:1.
78. A composition according to one of the preferred embodiments 1 through 44, comprising between 55% and 95% EPA.
79. A composition according to one of the preferred embodiments 1 through 44, comprising between 60% and 95% EPA.
80. A composition according to one of the preferred embodiments 1 through 44, comprising between 65% and 95% EPA.
81. A composition according to one of the preferred embodiments 1 through 44, comprising between 70% and 95% EPA.
82. A composition according to one of the preferred embodiments 1 through 44, comprising between 75% and 95% EPA.
83. A composition according to one of the preferred embodiments 1 through 44, comprising between 80% and 95% EPA.
84. A composition according to one of the preferred embodiments 1 through 44, comprising between 85% and 95% EPA.
85. A composition according to one of the preferred embodiments 1 through 44, comprising between 90% and 95% EPA.
86. A composition according to one of the preferred embodiments 1 through 44, comprising between 1% and 3% DPA.
87. A composition according to one of the preferred embodiments 1 through 44, comprising between 1% and 5% DPA.
88. A composition according to one of the preferred embodiments 1 through 44, comprising between 2% and 10% DPA.
89. A composition according to one of the preferred embodiments 1 through 44, comprising between 3% and 20% DPA.
90. A composition according to one of the preferred embodiments 1 through 44, comprising between 3% and 30% DPA.
91. A composition according to one of the preferred embodiments 1 through 44, comprising between 3% and 50% DPA.
92. A composition according to one of the preferred embodiments 1 through 44, comprising between 3% and 75% DPA.
93. A composition according to one of the preferred embodiments 1 through 44, comprising between 3% and 90% DPA.
94. A fatty acid composition according to one of the preferred embodiments 1 through 93, in which the fatty acids are present as ethyl esters.
95. A fatty acid composition according to one of the preferred embodiments 1 through 93, in which the fatty acids are present as free fatty acids.
96. A fatty acid composition according to one of the preferred embodiments 1 through 93, in which the fatty acids are present as esters in di-glyceride form.
97. A fatty acid composition according to one of the preferred embodiments 1 through 93, in which the fatty acids are present as esters in triglyceride form.
98. A fatty acid composition according to one of the preferred embodiments 94 through 97, also comprising a suitable anti-oxidant in a concentration sufficient to protect the fatty acids of the composition from oxidation.
99. A pharmaceutically suitable formulation comprising one of the compositions according to preferred embodiments 94 through 98, in which the amount of eicosapentaenoic acid plus docosapentaenoic acid is present in an amount between 100 and 10,000 mg.
100. A pharmaceutically suitable formulation or dosage form comprising one of the compositions according to preferred embodiments 94 through 98, in which the amount of eicosapentaenoic acid plus docosapentaenoic acid is present in an amount between 250 and 1,250 mg.
101. A pharmaceutically suitable formulation or dosage form comprising one of the compositions according to preferred embodiments 94 through 98, in which the amount of eicosapentaenoic acid plus docosapentaenoic acid is present in an amount between 500 and 1,100 mg.
102. A pharmaceutically suitable formulation or dosage form comprising one of the compositions according to preferred embodiments 94 through 98, in which the amount of eicosapentaenoic acid plus docosapentaenoic acid is present in an amount between 100 and 10.000 mg.
103. A method of administration or treatment to a subject of a formulation or dosage form according to one of the preferred embodiments 94 through 102 at a daily dose between 100 and 10,000 mg.
104. A method of administration or treatment to a subject of a formulation or dosage form according to one of the preferred embodiments 94 through 102 at a daily dose between 500 and 5,000 mg.
105. A method of administration or treatment to a subject of a formulation or dosage form according to one of the preferred embodiments 94 through 102 at a daily dose between 1,500 and 4,100 mg.
106. A method of treatment according to preferred embodiments 103 through 105, in which the subject is a patient diagnosed with very high triglycerides (equal or more than 500 mg/dL).
107. A method of treatment according to preferred embodiments 103 through 105, in which the subject is a patient diagnosed with high triglycerides (equal to or more than 200 mg/dL but less than 500 mg/dL).
108. A method of treatment according to preferred embodiments 103 through 105, in which the subject is a patient already undergoing treatment with a statin and then diagnosed with high triglycerides (equal to or more than 200 mg/dL but less than 500 mg/dL).
109. A method of treatment according to preferred embodiments 103 through 105, in which the subject is a patient diagnosed with mixed dyslipidemia with TG 200-499 mg/dL and LDL-cholesterol equal to or more than 190 mg/dL.
110. A method of treatment according to preferred embodiments 103 through 105, in which the subject is a patient diagnosed with mixed dyslipidemia with TG 300-700 mg/dL and LDL-cholesterol equal to or more than 190 mg/dL.
111. A method of treatment according to preferred embodiments 103 through 105, in which the subject is a patient diagnosed with mixed dyslipidemia with TG 200-499 mg/dL and non-HDL-cholesterol equal to or more than 200 mg/dL.
112. A method of treatment according to preferred embodiments 103 through 105, in which the subject is a patient diagnosed with mixed dyslipidemia with TG 300-700 mg/dL and non-HDL-cholesterol equal to or more than 200 mg/dL.
113. A method of treatment according to preferred embodiments 103 through 105, in which the subject is a patient diagnosed with mixed dyslipidemia with TG 200-499 mg/dL and LDL-cholesterol equal to or more than 160 mg/dL.
114. A method of treatment according to preferred embodiments 103 through 105, in which the subject is a patient diagnosed with mixed dyslipidemia with TG 300-700 mg/dL and LDL-cholesterol equal to or more than 160 mg/dL.
115. A method of treatment according to preferred embodiments 103 through 105, in which the subject is a patient diagnosed with mixed dyslipidemia with TG 200-499 mg/dL and non-HDL-cholesterol equal to or more than 160 mg/dL.
116. A method of treatment according to preferred embodiments 103 through 105, in which the subject is a patient diagnosed with mixed dyslipidemia with TG 300-700 mg/dL and non-HDL-cholesterol equal to or more than 160 mg/dL.
117. A method of treatment according to preferred embodiments 103 through 105, in which the subject is a patient diagnosed with mixed dyslipidemia with TG 200-499 mg/dL and LDL-cholesterol equal to or more than 130 mg/dL.
118. A method of treatment according to preferred embodiments 103 through 105, in which the subject is a patient diagnosed with mixed dyslipidemia with TG 300-700 mg/dL and LDL-cholesterol equal to or more than 130 mg/dL.
119. A method of treatment according to preferred embodiments 103 through 105, in which the subject is a patient diagnosed with mixed dyslipidemia with TG 200-499 mg/dL and non-HDL-cholesterol equal to or more than 130 mg/dL.
120. A method of treatment according to preferred embodiments 103 through 105, in which the subject is a patient diagnosed with mixed dyslipidemia with TG 300-700 mg/dL and non-HDL-cholesterol equal to or more than 130 mg/dL.
121. A method of treatment according to preferred embodiments 103 through 105, in which the subject is a patient diagnosed/assessed to be at substantially elevated risk for cardiovascular events.
122. A method of treatment according to preferred embodiments 103 through 105, in which the subject is a patient diagnosed with diabetes.
123. A method of treatment according to preferred embodiments 103 through 105, in which the subject is a patient diagnosed with pre-diabetes or metabolic syndrome.
124. A method of treatment according to one of the preferred embodiments 103 through 123, in which the treatment results in significant reduction of blood, serum or plasma triglyceride levels.
125. A method of treatment according to one of the preferred embodiments 103 through 123, in which the treatment results in significant reduction of blood, serum or plasma triglyceride levels while not significantly increasing blood, serum or plasma LDL-cholesterol levels.
126. A method of treatment according to one of the preferred embodiments 103 through 123, in which the treatment results in significant reduction of blood, serum or plasma total-cholesterol levels.
127. A method of treatment according to one of the preferred embodiments 103 through 123, in which the treatment results in significant reduction of blood, serum or plasma non-HDL-cholesterol levels.
128. A method of treatment according to one of the preferred embodiments 103 through 123, in which the treatment results in significant reduction of blood, serum or plasma LDL-cholesterol levels.
129. A method of treatment according to one of the preferred embodiments 103 through 123, in which the treatment results in significant reduction of blood, serum or plasma VLDL-cholesterol levels.
130. A method of treatment according to one of the preferred embodiments 103 through 123, in which the treatment results in significant reduction of blood, serum or plasma VLDL-cholesterol levels while not significantly increasing blood, serum or plasma LDL-cholesterol levels.
131. A method of treatment according to one of the preferred embodiments 103 through 123, in which the treatment results in significant reduction of blood, serum or plasma apo-B levels.
132. A method of treatment according to one of the preferred embodiments 103 through 123, in which the treatment results in significant reduction of blood, serum or plasma apo-C-III levels.
133. A method of treatment according to one of the preferred embodiments 103 through 123, in which the treatment results in significant reduction of blood, serum or plasma LP-PLA2 levels
134. A method of treatment according to one of the preferred embodiments 103 through 123, in which the treatment results in significant reduction of blood, serum or plasma hs-CRP levels.
135. A method of treatment according to one of the preferred embodiments 103 through 123, in which the treatment results in significant increase of blood, serum or plasma HDL-cholesterol levels.
136. A method of treatment according to one of the preferred embodiments 103 through 123, in which the treatment results in significant increase of blood, serum or plasma apo-A levels.
137. A method of treatment according to one of the preferred embodiments 103 through 123, in which the treatment results in significant reduction of the risk of suffering certain cardiovascular events.

What is claimed:

1. A pharmaceutical composition comprising docosapentaenoic acid (DPA) and docosahexaenoic acid (DHA) in an amount of at least 60% of the total amount of the fatty acids present in the composition, wherein the ratio of DPA to DHA (DPA:DHA) is between about 10:1 to about 1:10.

2. The pharmaceutical composition of claim 1, wherein the composition comprises DPA and DHA in an amount of at least 75% of the total amount of the fatty acids present in the composition.

3. The pharmaceutical composition of claim 1, wherein the composition comprises DPA and DHA in an amount of at least 80% of the total amount of the fatty acids present in the composition.

4. The pharmaceutical composition of claim 1, wherein the ratio of DPA to DHA (DPA:DHA) is between about 5:1 to about 1:5.

5. The pharmaceutical composition of claim 1, wherein the ratio of DPA to DHA (DPA:DHA) is between about 2:1 to about 10:1.

6. The pharmaceutical composition of claim 1, wherein the ratio of DPA to DHA (DPA:DHA) is between about 2:1 to about 8:1.

7. The pharmaceutical composition of claim 1, wherein the ratio of DPA to DHA (DPA:DHA) is between about 2:1 to about 5:1.

8. The pharmaceutical composition of claim 1, wherein the ratio of DPA to DHA (DPA:DHA) is between about 3:1 to about 5:1.

9. The pharmaceutical composition of claim 1, wherein the ratio of DHA to DPA (DHA:DPA) is between about 2:1 to about 10:1.

10. The pharmaceutical composition of claim 1, wherein the ratio of DHA to DPA (DHA:DPA) is between about 2:1 to about 8:1.

11. The pharmaceutical composition of claim 1, wherein the ratio of DHA to DPA (DHA:DPA) is between about 2:1 to about 5:1.

12. The pharmaceutical composition of claim 1, wherein the ratio of DHA to DPA (DHA:DPA) is between about 3:1 to about 5:1.

13. A method of treating, preventing, reducing the occurrence of, and improving symptoms associated with a liver-related disease or condition in a subject in need thereof, comprising administering to the subject a composition of claim 1.

14. The method of claim 13, wherein the liver-related disease or condition is selected from the group consisting of: fatty liver, non-alcoholic steatohepatitis (NASH), non-alcoholic fatty liver disease (NAFLD), alcoholic steatohepatitis (ASH), hepatitis, HIV (human immunodeficiency virus) infection, drug-induced fatty liver or sequelae, liver failure, liver transplantation, transplanted liver failure, liver damage associated with renal failure or disease, abnormally elevated liver enzymes, or type 2 diabetes.

15. A method of reducing expression of HMG-CoA reductase in a subject in need thereof, comprising administering to the subject a composition of claim 1.

16. A method of reducing levels of HMG-CoA reductase in a subject in need thereof, comprising administering to the subject a composition of claim 1.

17. A method of reducing the expression of proprotein convertase subtilisin/kexin type 9 (PCSK9) in a subject in need thereof, comprising administering to the subject a composition of claim 1.

18. A method of reducing levels of proprotein convertase subtilisin/kexin type 9 (PCSK9) in a subject in need thereof, comprising administering to the subject a composition of claim 1.

19. A method of treating, preventing, reducing the occurrence of, and improving symptoms associated with a liver-related disease or condition in a subject in need thereof, comprising administering to the subject a composition comprising docosapentaeoic acid (DPA) and docosahexaenoic acid (DHA) in a daily dosage amount of between 200 mg/day and 8,100 mg/day.

20. The method of claim 1, wherein the composition comprises docosapentaeoic acid (DPA) and docosahexaenoic acid (DHA) in a daily dosage amount of between 300 mg/day and 6,100 mg/day.
